(19) United States
(12) Patent Application Publication
(10) Pub. No.: US 2012/0315629 A1
(43) Pub. Date: Dec. 13, 2012

(54) Cloning and Expression of arNOX Protein Transmembrane 9 Superfamily (TM9SF), Methods and Utility

(75) Inventors: D. James Morre, West Lafayette, IN (US); Xiaoyu Tang, West Lafayette, IN (US); Sara Dick, Valparaiso, IN (US); Christiaan Meadows, Lafayette, IN (US); Dorothy M. Morre, West Lafayette, IN (US)

(73) Assignee: Nox Technologies ,Inc., Malvern, PA (US)

(21) Appl. No.: 13/390,795

(22) PCT Filed: Aug. 17, 2010
(86) PCT No.: PCT/US2010/045745

§ 371 Date: Aug. 22, 2012

Related U.S. Application Data
(60) Provisional application No. 61/234,368, filed on Aug. 17, 2009.

(51) Int. Cl. C12N 9/02; C12N 1/21; C07K 16/40; C12N 1/19; C12Q 1/68; G01N 33/573; C12N 15/53; C12N 5/10

(52) U.S. Cl. 435/6.11; 536/23.2; 435/252.33; 435/325; 435/358; 435/254.2; 435/189; 435/6.12; 435/7.4; 530/387.9

(57) ABSTRACT

Described are cell surface and circulating markers for aging related disorders (specific isoforms of NADH oxidase (arNOX)). Recombinant age-related NADH oxidase isoforms and their coding sequences and methods for detecting arNOX isoform presence and quantitation in tissues and in blood, sera, urine, saliva, perspiration and in other body fluids, are provided. Recombinant arNOX proteins are useful in preparing antigens for use in the generation of monoclonal and polyclonal antibodies as well as immunogenic compositions for diagnosis and treatment of aging disorders. DNA probes based on the DNA sequence information provide may be used to identify individuals at risk for aging disorders and for development of therapeutic interventions or anti-aging cosmetic or other formulations of benefit in slowing the aging process in mammals.

### CROSS-REFERENCE TO RELATED APPLICATIONS

This application claims benefit of U.S. Provisional Application 61/234,368 filed Aug. 17, 2009, which is incorporated by reference herein to the extent there is no inconsistency with the present disclosure.

### BACKGROUND

This disclosure relates to the area of molecular biology and biochemistry, in particular, as related to prevention or treatment of disorders caused by oxidative damage by aging-specific isoforms of NADH oxidase (arNOX) and as a circulating marker for aging-related disorders, recombinant expression and screening assays for expression or inhibitors thereof.

A cell surface protein with hydroquinone (NADH) oxidase activity (designated NOX) that functions as a terminal oxidase of plasma membrane electron transport to complete an electron transport chain involving a cytosolic hydroquinone reductase, plasma membrane located quinones and the NOX protein was elucidated by the Inventors (Kishi et al., 1999, Biochem. Biophys. Acta 1412:66-77 and Morré, 1998, Plasma Membrane Redox Systems and their Role in Biological Stress and Disease, Klewer Academic Publishers, Dordrecht, The Netherlands, pp. 121-156). This system provides a rational basis for operation of the mitochondrial theory of aging and for propagation of aging related mitochondrial lesions, including a decline in mitochondrial ATP synthetic capacity and other energy-dependent processes during aging (Boffoli et al., 1996, Biochem. Biophys. Acta 1226:73-82; Lenaz et al., 1998, BioFactors 8:195-204; de Grey, 1997, BioEssays 19:161-166; and de Grey, 1998, J. Anti-Aging Med. 1:53-66).

The plasma membrane NADH oxidase (NOX or ENOX) is a unique cell surface protein with hydroquinone (NADH) oxidase and protein disulfide-thiol interchange activities that normally responds to hormone and growth factors. arNOX (or ENOX3) are a family of growth related proteins that are associated with aging cells.

The aging-related isoform of NADH oxidase (arNOX) is a member of this family of ENOX proteins. The circulating form of arNOX increases markedly in human sera and in lymphocytes of individuals, especially after the age of 65. The arNOX protein is uniquely characterized by an ability to generate superoxide radicals, which may contribute significantly to aging-related changes including atherogenesis and other action-at-a-distance aging phenomena. Activity of arNOX in aging cells and in sera has been described previously (Morré and Morré, 2006, Rejuvenation Res. 9:231-236).

Aging has been proposed to result from an ever-increasing level of destructive chemical reactions involving free radicals, with mitochondria as the principal mediators of the process (Harman, 1956, J. Gerontol. 11:298-300 and Harman, 1972, J. Am. Geriatr. Soc. 20:145-147). The main line of reasoning to support this ideas is that, of all subcellular components, mitochondria is both a major source of free radicals and a major direct victim of free radical damage. As a result, loss of mitochondrial function may be the driving intracellular change underlying aging, and the cause of other pro-oxidant changes such as slower protein turnover. There is considerable indirect as well as direct experimental support for the theory. For example, a decline in ATP synthesis capacity and of energy-depending processes during aging has been reported (Syrovy and Gutmann, 1997, Exp. Gerontol. 12:31-35; Sugiyama et al., 1993, Biochem. Mol. Biol. Intl. 30:937-944; Boffoli et al., 1996, Biochim. Biophys. Acta 1226:73-82; and Lenaz et al., 1998, BioFactors 8:195-204).

This model of the effects of arNOX is consistent with the Mitochondrial Theory of Aging, which holds that during aging, increased reactive oxygen species in mitochondria cause mutations in the mitochondrial DNA and damage mitochondrial components, resulting in senescence. The mitochondrial theory of aging proposes that accumulation of spontaneous somatic mutations of mitochondrial DNA (mtDNA) leads to errors of mtDNA encoded polypeptide chains (Manczak M et al., 2005, J. Neurochem. 92(3):494-504). These errors, occurring in mtDNA encoded polypeptide chains, are stochastic and randomly transmitted during mitochondrial and cell division. The consequence of these alterations is defective oxidative phosphorylation. Respiratory chain defects may become associated with increased oxidative stress amplifying the original damage (Ozawa, 1995, Biochim. Biophys. Acta 1271:177-189; and Lenaz, 1998, Biochim. Biophys. Acta 1366:53-67). In this view, therefore, mutated mitochondrial DNA, despite being present only in very small quantities in the body, may be the major generator of oxidative stress.

Where accumulation of somatic mutations of mtDNA leads to defective oxidative phosphorylation, a plasma membrane oxido-reductase (PMOR) system has been suggested to augment survival of mitochondrially deficient cells through regeneration of oxidized pyridine nucleotide (de Grey, 1997, BioEssays 19:161-166; de Grey, 1998, Anti-Aging Med. 1:53-66; Yoneda et al., 1995, Biochem. Biophys. Res. Comm. 209:723-729; Schon et al., 1996, Cellular Aging and Cell Death, Wiley and Sons, New York, pp. 19-34; Ozawa et al., 1997, Physiol. Rev. 77:425-464; and Lenaz, 1998, BioFactors 8:195-204). However, alterations of mtDNA of themselves have been difficult to link to other forms of cellular and tissue changes related to aging. Chief among these is low density lipoprotein (LDL) oxidation and atherogenesis (Steinberg, 1997, J. Biol. Chem. 272:20963-20966).

A model to link accumulation of lesions in mtDNA to extracellular responses, such as the oxidation of lipids in low density lipoprotein (LDLs) and the attendant arterial changes, was first proposed with rho° cells (Larm et al., 1994, Biol. Chem. 269:30097-30100; Lawen et al., 1994, Mol. Aspects. Med. 15:s13-s27; de Grey, 1997, BioEssays 19:161-166; and de Grey, 1998, Anti-Aging Med. 1:53-66). Similar studies have been conducted with transformed human cells in culture (Vaillant et al., 1996, Bioenerg. Biomemb. 28:531-540).

Under conditions where plasma membrane oxidoreductase (PMOR) is overexpressed, electrons are transferred from NADH to external acceptors by a defined electron transport chain, resulting in the generation of reactive oxygen species (ROS) at the cell surface. Such cell surface-generated ROS may then propagate an aging cascade originating in mitochondria to both adjacent cells as well as to circulating blood components such as low density lipoproteins (Morré and Morré, 2006, Rejuvenation Res. 9:231-236).

Because aging poses a significant threat to human health and because aging-related disorders result in significant economic and social costs, there is a long-felt need in the art for effective, economical and technically simple systems in which to assay for or model inhibitors of aging-related disease states, for aging-related, enzyme specific markers and antibodies, and for reagents, inhibitor and activator screening methods and expression systems.

### SUMMARY

It is an object to provide recombinant age-related NADH oxidase isoforms (termed arNOX herein) as recombinant membrane-bound proteins or as soluble proteins, their coding sequences and isolated host cells containing these sequences and expressing these proteins. The full length sequences have specifically exemplified genomic coding sequences as given in Table 1 and in SEQ ID NOs:1, 3, 5, 7 and 9. The Sequence Listing includes information for the corresponding spliced coding sequences. The full length proteins have amino acid sequences as given in Table 2 and in SEQ ID NOs:2, 4, 6, 8 and 10. Also encompassed within this object are coding sequences which are synonymous with those specifically exemplified sequences. A further aspect of the recombinant arNOX proteins are those for soluble (truncated) arNOX, as shown in Tables 3 and in SEQ ID NOs:13-17. Those truncated proteins lack the C-terminal portions which define the membrane-integrating region. Optionally, the recombinant arNOX proteins may further comprise “tag” regions to facilitate purification after expression tag sequences which are well known to the art, and they include hexahistidine, flagellar antigen (Flag), glutathione synthetase (GST), biotin-binding peptide (AviTag), and others.

Also contemplated are sequences which encode an aging cell surface marker and which coding sequences hybridize under stringent conditions to the specifically exemplified full length or partial sequences and which have the enzymatic activity of arNOX. The cell surface arNOX is characteristic of advancing age, and when shed from the cell surface, it circulates in body fluids as a non-invasive marker of aging disorders. The recombinant arNOX proteins, especially the enzymatically active portions of the full length protein, are useful in preparing antigens for use in generation of both polyclonal and monoclonal antibodies for diagnosis and treatment of aging disorders.

Further provided are methods for determining aging-related arNOX in a mammal, said methods comprising the steps of detecting the presence and quantitation of one or more arNOX isoforms in a biological sample, by measurement of particular proteins by measurement of enzymatic activity, immunological detection methods or by measurement of the transcriptional expression of the relevant genes.

The present disclosure enables the generation of antibody preparations, especially using a recombinant arNOX isoform or a truncated arNOX isoform protein or an antigenic peptide derived in sequence from an arNOX isoform amino acid sequence, which antibody specifically binds to an protein selected from the group consisting of a protein characterized by amino acid sequences as given in SEQ ID NOs:2, 4, 6, 8, 10 or 13-17 or a peptide sequences as set forth herein. These antibody-containing compositions are useful in detecting one or more arNOX proteins in blood, serum, saliva, perspiration or tissue from a patient (a biological sample) to validate arNOX status and/or response to therapeutic intervention.

Immunogenic compositions comprising at least one recombinant arNOX isoform or a truncated arNOX isoform protein or an antigenic peptide derived in sequence from an arNOX isoform amino acid sequence, which specifically binds to an antibody selected from the group consisting of a protein characterized by amino acid sequences as given in Table 2. Peptides useful for generating antibodies specific to each of the 5 arNOX isoforms have amino acid sequences as follows: TM9SF1a and/or TM9SF1b, QETYHYYQLPVCCPEKIRHKSLSLGEVLDGDR, amino acids 56-87 of SEQ ID NO:2; TM9SF2, VLPYEYTAFDFCQASEGKRPSENLGQVLFGER, amino acids 73-104 of SEQ ID NO:6; TM9SF3, QETYKYFSLPFCVGSKKSISHYHETLGEALQGVE, amino acids 55-88 of SEQ ID NO:8; and TM9SF4, QLPYEYYSLPFCQPSKITYKAENLGEVLRGDR, amino acids 53-84 of SEQ ID NO:10 are useful for preparing antibodies as described above. Antibody specific to the membrane-bound form of TM9SF1a (but not also to TM9SF1b) is made using a peptide antigen with the sequence set forth in amino acids 548-568 of SEQ ID NO: 2 (LYSVFYYARRSNMSGAVQTVE). Immunogenic compositions with peptide antibodies typically comprise the peptide bound to a carrier molecule, which may be keyhole limpet hemocyanin, among other proteins as well known to the art. In addition, such immunogenic compositions may be used to reduce the severity of certain deleterious aspects of oxidation reactions carried out by the arNOX enzymes in a human or animal, thereby improving the health and well-being of the individual to which such an immunogenic composition has been administered.

Antibodies specific for arNOX and the shed (forms of soluble) arNOX in tissues and in the urine and serum, perspiration, saliva or other body fluids are useful, for example, as probes for screening DNA expression libraries or for detecting or diagnosing aging-related disorder or tendency for such a disorder in a sample from a human or animal. Desirably the antibodies (or second antibodies which are specific for the antibody which recognizes arNOX) are labeled by joining, either covalently or noncovalently, a substance which provides a detectable signal. Suitable labels include but are not limited to radionuclides, enzymes, substrates, cofactors, inhibitors, fluorescent agents, chemiluminescent agents, magnetic particles and the like. United States patents describing the use of such labels include, but are not limited to, U.S. Pat. Nos. 3,817,837; 3,580,752; 3,939,350; 3,996,345; 4,277,437; 4,275,149; and 4,366,241. Antibodies useful in diagnostic and screening assays can be prepared using a peptide antigen whose sequence is derived from all or a part of the full length protein or a protein corresponding to am amino acid sequence among those given in Table 2 or 3.

Immunogenic compositions and/or vaccines comprising an arNOX protein or antigenic portion thereof, such as a peptide as described herein above, may be formulated and administered by any means known in the art. They are typically prepared as injectables, either as liquid solutions or suspensions. Solid forms suitable for solution in, or suspension in, liquid prior to injection may also be prepared. The preparation may also, for example, be emulsified, or the protein(s)/peptide(s) encapsulated in liposomes. Advantageously, such an immunogenic composition comprises at least one component which stimulates an immune response, for example, an adjuvant. Administration of an immunogenic composition can be via subcutaneous, intradermal, intraperitoneal, intravenous, intramuscular route in a human or experimental animal, or into a footpad of an experimental animal, or other route known to the art.

Northern blot analyses may be used to indicate that the coding sequence(s) of arNOX is (are) expressed in individuals at risk for aging disorders. The availability of the sequence(s) makes possible rapid further testing of the specificity of expression and future development of therapeutic interventions or antiaging cosmetic or other formulations.

The nucleotide sequences encoding human arNOX, recombinant human arNOX proteins and recombinant cells which express recombinant human arNOX can be used in the production of recombinant arNOX protein(s) or portions thereof for use in aging diagnostic protocols and in screening assays to identify new anti-aging drugs and/or nutritional supplements, cosmeceuticals, nutriceuticals and aging prevention or retardation strategies.

### BRIEF DESCRIPTION OF THE DRAWING

FIG. 1 is a diagrammatic representation of the membrane association of the TM-9 Protein Superfamily members. An N-terminal soluble fragment is proteolytically cleaved at the cleavage site and released into the exterior milieux of the cells or into the lumens of endocytic vesicles.

FIG. 2 illustrates the identities and positions of functional arNOX motifs of isoform SF2. See SEQ ID NO:6 for the amino acid sequence of the soluble enzyme.

FIG. 3 illustrates arNOX activity of recombinant soluble arNOX isoform SF-4 showing only superoxide generation as measured by reduction of ferricytochrome c. Maxima are separated by intervals of 26 min.

FIG. 4 illustrates arNOX activity of recombinant soluble arNOX isoform SF-4 showing the typical 5-peak pattern of activity characteristic of ENOX proteins in general. Note that the units of specific activity are μmoles/min/mg protein. Superoxide generation is intensified with maximum 3 of the 5-maxima oscillatory pattern.

FIG. 5 illustrates induction of arNOX activity in lymphocytes from a 28 yr old female at day 2 and at days 5 and 6 of incubation at 8° C. Note the marked inductions of all five arNOX isoforms within this time period.

FIG. 6 shows the time course of induction of arNOX activity (upper panel) and arNOX messenger RNA (lower panel). The latter compares the results obtained using lymphocytes from 22 and 73 yr old individuals

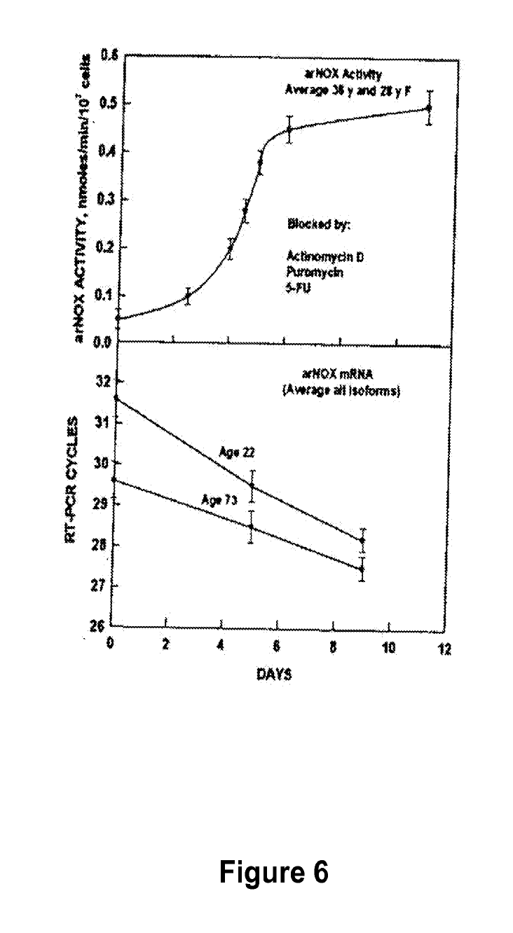

FIG. 7 illustrates the results obtained with the sequential addition of peptide antibodies to sera to show identification of association of specific maxima present in the prebleed with specific isoforms SF1 to SF4. After addition of SF-4-specific antibody, no evidence of any remaining arNOX activity was observed. After addition of antibody specific to TM9SF3, only one isoform remained. After addition of TM9SF2-specific antibody, two isoforms remained, etc.

FIG. 8 shows arNOX detection and relative amounts via ELISA in skin, saliva and serum using arNOX-specific antibodies prepared using arNOX-specific peptides as antigens.

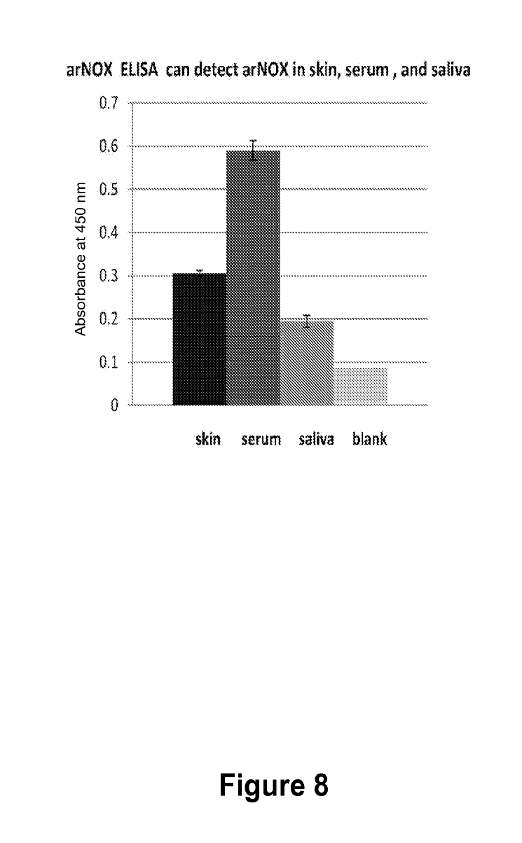

FIGS. 9A-9C show relative amounts of arNOX in materials from older and younger persons, as estimated using ELISA with an arNOX-specific antibody preparation. FIG. 9A shows the results for arNOX in skin filings; FIG. 9B shows relative amounts in serum samples taken from four individuals of three different ages, and FIG. 9C shows the results for ELISAs carried out using a combination of antibodies specific to all arNOX isoforms in saliva samples from older and younger individuals.

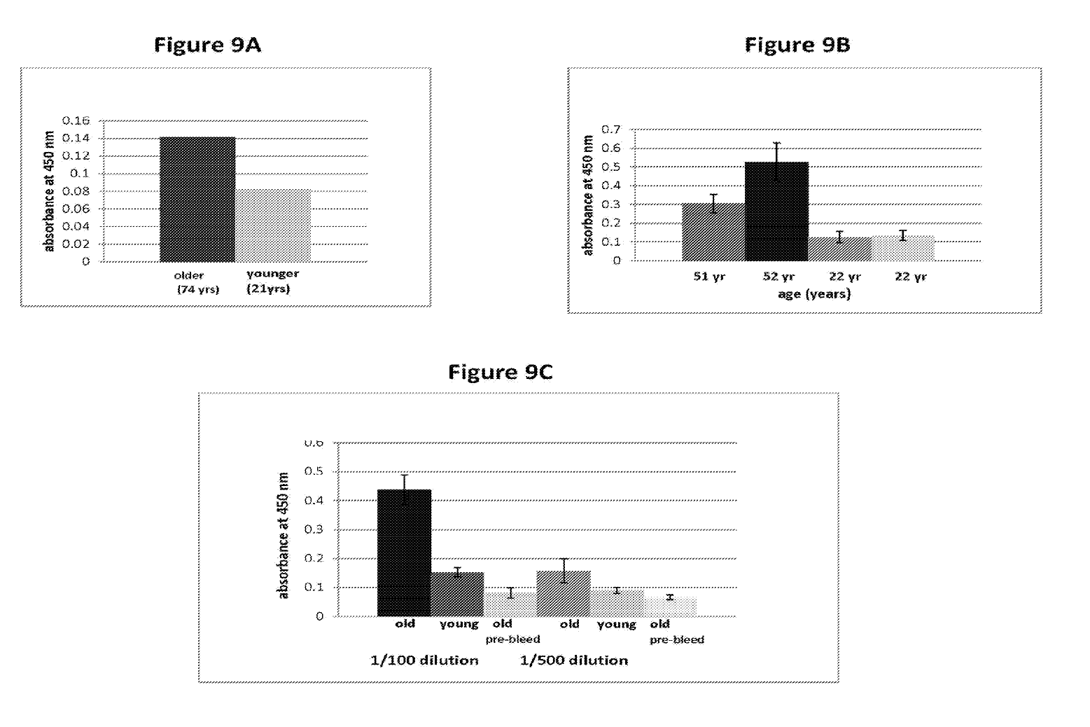

### DETAILED DESCRIPTION

As used herein, the term “disorder” refers to an ailment, disease, illness, clinical condition, or pathological condition.

As used herein, the term “reactive oxygen species” refers to oxygen derivatives from oxygen metabolism or the transfer of free electrons, resulting in the formation of free radicals (e.g., superoxides or hydroxyl radicals).

As used herein, the term “antioxidant” refers to compounds that neutralize the activity of reactive oxygen species or inhibit the cellular damage done by said reactive species.

As used herein, the term “transmembrane 9 super family” refers to any and all proteins with sequence similarity or homology to members 1a, 1b, 2, 3 and 4 as presented in Tables 1 and 2 herein, also known collectively as arNOX or arNOX proteins.

As used herein, the term “isolated host cell” means that the cell is not part of an intact multicellular organism.

The association of the Transmembrane 9 (TM-9) Superfamily of proteins with what was assayed as arNOX activity began with analyses of yeast deletion and over expression strains of Saccharomyces cerevisiae. An arNOX activity was identified in a deletion library, and the respective deletion was traced to gene YErII3C; the corresponding protein was then characterized from a yeast overexpression library and determined to be a member of the Transmembrane 9 Superfamily. An expressed sequence tag (EST) in the yeast database permitted identification of human arNOX from a homology search of the human genome. The human arNOX cDNA encodes a polypeptide having a highly hydrophobic C-terminal portion organized into nine transmembrane domains with a very similar structure and sequence to members of a novel family of multispanning domain proteins designated “TM9SF” (transmembrane protein 9 superfamily) by the Human Gene Nomenclature Committee. The leader member of the TM9SF family is the Saccharomyces cerevisiae EMP70 gene product, a 70 kDa precursor that is processed into a 24 kDa protein (p24a) located in the endosomes (Singer-Kruger et al., 1993, J. Biol. Chem. 268: 14376-14386). To date, five subtypes (isoforms) of human TM9SF proteins have been identified, i.e., TM9SF-I (hMP70; Chuluba de Tapia et al., 1997, Gene 197: 195-204), TM9SF-Ib, TM9SF-2 (p76; Schimmoller et al., 1998, Gene 216: 311-318), TM9SF-3 and D87444, which exhibit 30-40% amino acid sequence identity to each other and with the yeast p24a precursor (Sugasawa et al., 2000, Gene 273: 229-237). All of the isoforms exhibit arNOX activity. This was a surprising result that arNOX activity was the result of at least five separate proteins.

Hydropathy analysis (Kyte and Doolittle, 1982, J. Mol. Biol. 157: 105-132) of p76 and its close relatives revealed that these proteins share a unique membrane binding domain (Schimmoller et al., 1998, Gene 216: 311-318). They also contain a short N-terminal hydrophobic extension characteristic of a signal sequence, followed by a mostly hydrophilic, amino terminal portion that extends up to amino-acid residue 300 in certain family members. The remaining portions of these proteins are extremely hydrophobic and contain nine transmembrane domains to make them integral membrane proteins that adopt a type 1 topology. Polypeptide translocation would be initiated via their N-terminal hydrophobic signal sequence and they would ultimately be anchored in the membrane via stop-transfer sequences.

The EMP70 gene was cloned based on the N-terminal sequence information obtained by microsequencing this 24 kDa protein (Singer-Kruger et al., 1993, J. Biol. Chem. 268: 1437614386; Genembl database entry X67316). Sequencing of the S. cerevisiae genome revealed that the EMP70 gene is located on chromosome XII (GenBank accession number U53880). The p76 cDNA encodes a protein of 663 amino acids and a predicted mass of 76 kDa (Gen Bank accession number U81006).

At the protein level, p76 and the p24a protein precursor (Emp70) share 35% amino acid sequence identity. Strikingly, the highest level of sequence identity is localized to the C-terminal 60% of these proteins; in contrast, the N-terminal domains show much greater amino acid sequence diversity. Another human homolog (GenBank accession D87444) has a predicted mass of 72 kDa and is referred to as human EMP70p, to distinguish it from p76.

Members of the TM-9 protein superfamily are all characterized as cell surface proteins (as are arNOX proteins) having a characteristic series of 9 membrane spanning hydrophobic helices that criss-cross the plasma membrane. The transmembrane regions are highly conserved and similar or identical in each of the five isoforms. There are 5 such isoforms known (1a, 1b, 2, 3 and 4; Isoforms 1a and Ib are very similar). They appear to be encoded by different genes. They are not splice variants. The TM-9 family members are known to be present on endosomes.

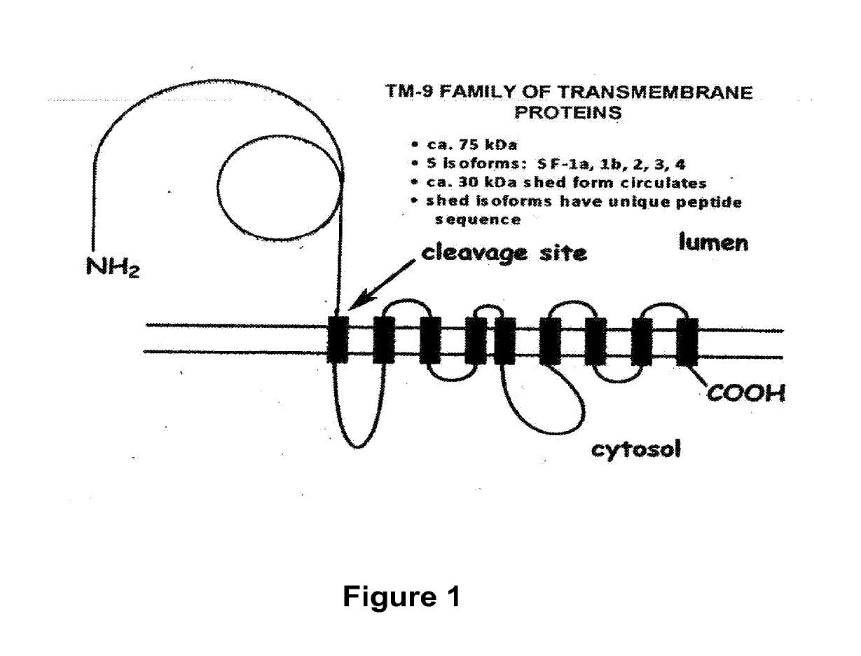

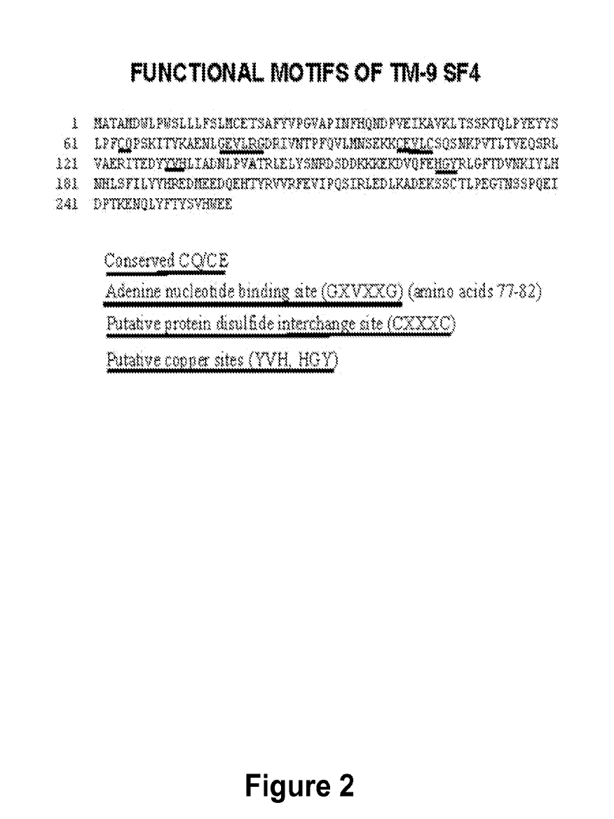

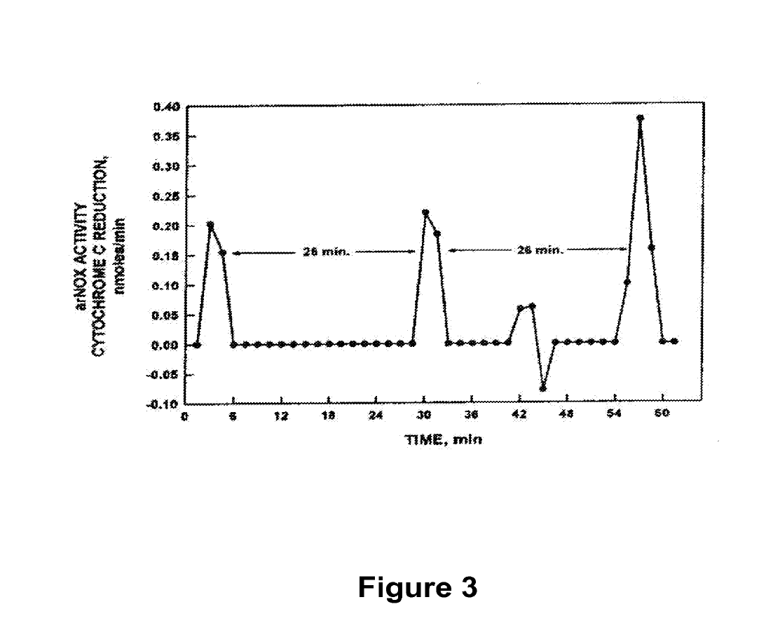

The present inventors discovered that the ca. 30 kDa N-terminal regions of the noted TM9SF proteins, which are exposed at the external surface of the plasma membrane, are shed into the blood and other body fluids (saliva, perspiration, urine); they are present in sera and plasma and are measured collectively as arNOX. All five isoforms are present in samples of aged individuals although in different ratios. There is a serine protease cleavage site at the arrow in FIG. 1. Each of the shed forms contains functional motifs required of an ENOX protein, and the functional motifs are unique to the arNOX family. The functional motifs are illustrated in FIGS. 2 and 3 as are the sequences of the soluble forms of the arNOX proteins isolated. Despite the presence of required functional motifs in each of the isoforms, sequence identify among the different isoforms was minimal. Their identification from amino acid sequence or on sequence analysis of soluble forms of arNOX would not have been obvious even to one skilled in the art.

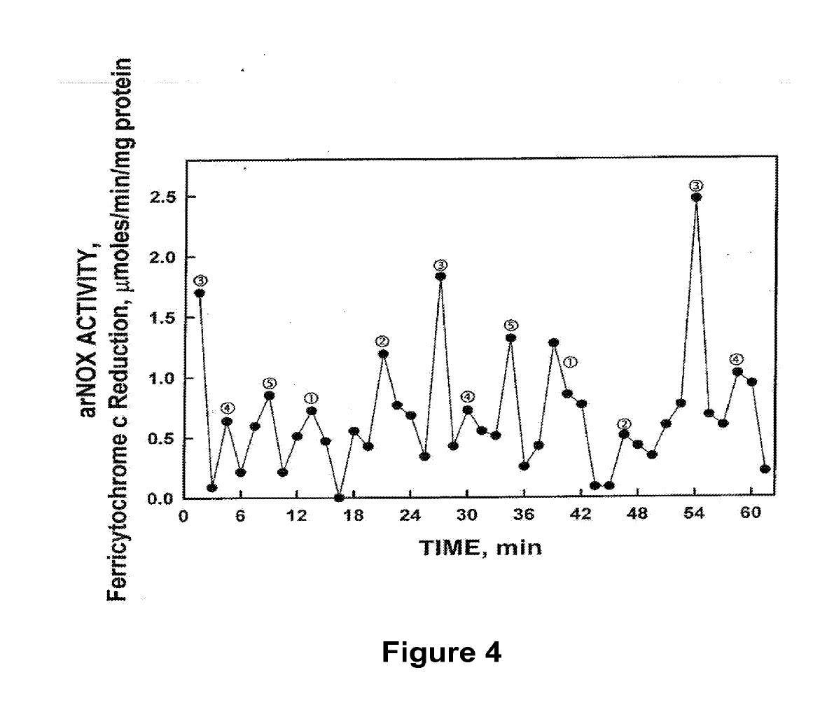

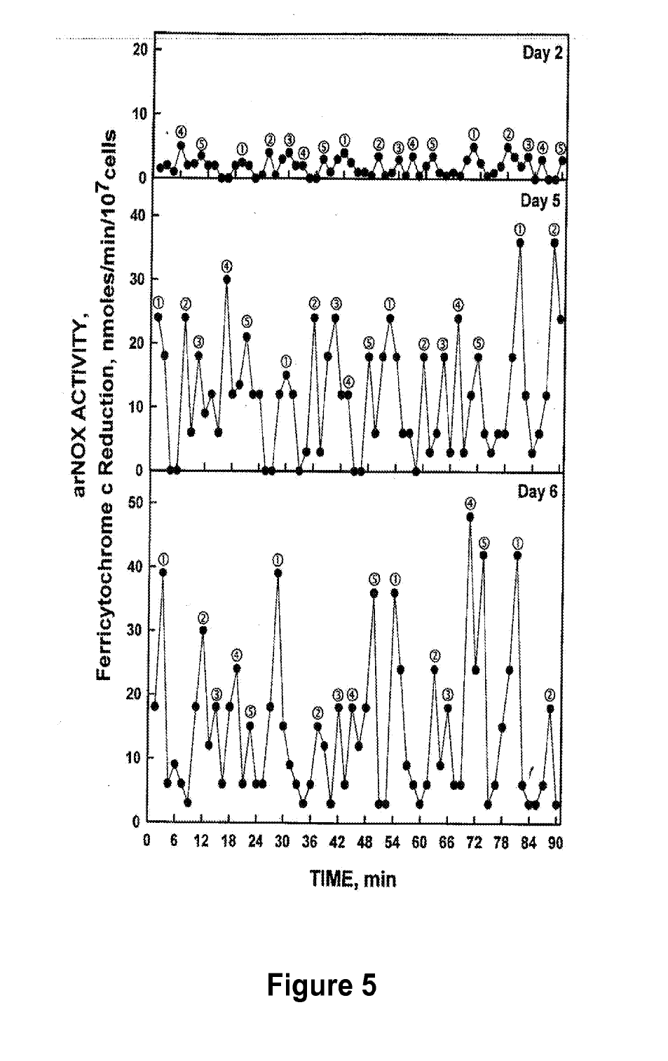

cDNA was obtained for the SF4 isoform and expression in yeast was attempted. Expression of the full length protein (SEQ ID NO:9) was not successful. However, cloning of the soluble fragment of TM9SF4 was successful, and the cloned protein had functional characteristics identical to those of an arNOX protein (FIGS. 4 and 5). The soluble amino acid and DNA sequences of the soluble forms of the isoforms were then utilized to prepare peptide antibodies to each of the isoforms and RNA probes to each of the isoforms, respectively. The antibodies were used to systematically identify each of the 5 isoforms in human sera and saliva to correspond to the known sequences of the TM-9 Super Family of protein isoforms, DNA sequence information was used to generate RT-PCR probes for each of the isoforms and demonstrate their expression in both human lymphocytes and human skin explants. These data confirm the TM9 superfamily of proteins as the genetic origins of the five known arNOX isoforms of human sera, plasma and other body fluids.

Current assays for arNOX are time consuming, inaccurate, and, while revealing five different isoforms, an activity maxima separated by intervals of 26 min, do not associate each maximum with a specific isoform. To avert these and other difficulties, the information disclosed herein has been used to develop ELISA-based assays for arNOX that are isoform specific. Peptide antibodies were generated in rabbits to the soluble protein sequence of each of the isoforms. An arNOX source was coated on each of 5 replicated wells of a 96 well ELISA plate and after appropriate washing and blocking, the isoform-specific antibodies were added singly to each of the 5 replicated wells or as a mixture if the objective was simply to measure total arNOX. A peroxidase-linked second antibody was added along with colorimetric substrate and the developed color determined in an automated plate reader. The absorbance readings were linear with arNOX amounts and quantitated by means of a standard curve using recombinant soluble arNOX protein generated as described herein. The ELISA protocol is standard and not unique. However, the use of antibodies to arNOX isoforms as a method of arNOX and arNOX isoform quantitation is new and novel and included here to further demonstrate nonobvious utility of these findings.

It has further been determined that the TM9SF isoforms are not uniformly distributed in body fluids, including serum. However, biological samples can be from a subject mammal of interest, especially a human, and can be, without limitation, a skin sample, saliva, blood, serum, urine, intraperitoneal fluid, tissue sample or other sample from a subject mammal.

It is understood by the skilled artisan that there can be limited numbers of amino acid substitutions in an arNOX protein without significantly affecting function, and that nonexemplified arNOX can have some amino acid sequence divergence from the specifically exemplified amino acid sequence(s). Such naturally occurring variants can be identified, e.g., by hybridization to the exemplified coding sequence (or a portion thereof capable of specific hybridization to human arNOX sequences) under conditions appropriate to detect at least about 70% nucleotide sequence homology, preferably about 80%, more preferably about 90% or 95-100% sequence homology, or any integer within an above specified range. Preferably the encoded arNOX has at least about 90%, or any integer between 90 and 100% amino acid sequence identity to the exemplified arNOX amino acid sequence(s). In examining nonexemplified sequences, demonstration of the characteristic arNOX activities and the sensitivity of those to arNOX-specific inhibitors such as salicin allow one of ordinary skill in the art to confirm that a functional arNOX protein is produced.

Also within the scope of the present disclosure are isolated nucleic acid molecules comprising nucleotide sequences encoding arNOX proteins and which hybridize under stringent conditions to a nucleic acid molecule comprising coding sequences within the nucleic acid sequences given in Table 1. DNA molecules with at least 85% nucleotide sequence identity to a specifically exemplified arNOX coding sequence of the present invention are identified by hybridization under stringent conditions using a probe as set forth herein. Stringent conditions involve hybridization at a temperature between 65° C. and 68° C. in aqueous solution (5×SSC, 5×Denhardt's solution, 1% sodium dodecyl sulfate) or at about 42° C. in 50% formamide solution, with washes in 0.2×SSC, 0.1% sodium dodecyl sulfate at room temperature, for example. The specifically exemplified arNOX sequences of the present invention are readily tested by an ordinary skill in the art.

TABLE 1
DNA sequences encoding arNOX transmembrane superfamily
genes, 1a, 1b, 2, 3 and 4

Transmembrane 9 superfamily member 1 isoform a (Homo sapiens)
VERSION NP_006396.2 GI:21361315
DBSOURCE REFSEQ: accession NM_006405.5
(SEQ ID NO: 1)

| | |
|---|---|
| 1 | ggccgcgctg ccgatcgccg ggaggacccc cgcctcgccg aagacgggcg gggcaagccg |
| 61 | agcctcacgg ggtccccgga gctgggccgg gcctccagat ggagaaggcg caacggggag |
| 121 | ttcttgagta agccagagcg gtgtccagcg cggtgtagcc gcagccgccg ctgtcaggcg |
| 181 | cagcaacggg caaccccgta gaagtcggtc ggcaggtcct ctccaacccg ccgctaccgc |
| 241 | gccgctgtgg gagagacccc agcaggagcc caaaggcagc tacgggggcg cgaaggccgc |
| 301 | tggcgccgcc tcggccagcc cttcccgcgc ggttccactg ccttaaggat gacagtcgta |
| 361 | gggaaccctc gaagttggag ctgccagtgg ttgccaatcc tgatactgtt gctgggcaca |
| 421 | ggccatgggc caggggtgga aggcgtgaca cactacaagg ccggcgaccc tgttattctg |
| 481 | tatgtcaaca aagtgggacc ctaccataac cctcaggaaa cttaccacta ctatcagctt |
| 541 | ccagtctgct gccctgagaa gatacgtcac aaaagcctta gcctgggtga agtgctggat |
| 601 | ggggaccgaa tggctgagtc tttgtatgag atccgctttc gggaaaacgt ggagaagaga |
| 661 | attctgtgcc acatgcagct cagttctgca caggtggagc agctgcgcca ggccattgaa |
| 721 | gaactgtact actttgaatt tgtggtagat gacttgccaa tccggggctt tgtgggctac |
| 781 | atggaggaga gtggtttcct gccacacagc cacaagatag gactctggac ccatttggac |
| 841 | ttccacctag aattccatgg agaccgaatt atatttgcca atgtttcagt gcgggacgtc |
| 901 | aagccccaca gcttggatgg gttacgacct gacgagttcc taggccttac ccacacttat |
| 961 | agcgtgcgct ggtctgagac ttcagtggag cgtcggagtg acaggcgccg tggtgacgat |
| 1021 | ggtggtttct ttcctcgaac actggaaatc cattggttgt ccatcatcaa ctccatggtg |
| 1081 | cttgtgtttt tactggtggg ttttgtggct gtcattctaa tgcgtgtgct tcggaatgac |
| 1141 | ctggctcggt acaacttaga tgaggagacc acctctgcag gttctggtga tgactttgac |
| 1201 | cagggtgaca atggctggaa aattatccat acagatgtct tccgcttccc cccataccgt |
| 1261 | ggtctgctct gtgctgtgct tggcgtgggt gcccagttcc tggcccttgg cactggcatt |
| 1321 | attgtcatgg cactgctggg catgttcaat gtgcaccgtc atggggccat taactcagca |
| 1381 | gccatcttgt tgtatgccct gacctgctgc atctctggct acgtgtccag ccacttctac |
| 1441 | cggcagattg gaggcgagcg ttgggtgtgg aacatcattc tcaccaccag tctcttctct |
| 1501 | gtgcctttct tcctgacgtg gagtgtggtg aactcagtgc attgggccaa tggttcgaca |
| 1561 | caggctctgc cagccacaac catcctgctg cttctgacgg tttggctgct ggtgggcttt |
| 1621 | cccctcactg tcattggagg catctttggg aagaacaacg ccagcccctt tgatgcaccc |
| 1681 | tgtcgcacca agaacatcgc ccgggagatt ccaccccagc cctggtacaa gtctactgtc |
| 1741 | atccacatga ctgttggagg cttcctgcct ttcagtgcca tctctgtgga gctgtactac |
| 1801 | atctttgcca cagtatgggg tcgggagcag tacactttgt acggcatcct cttctttgtc |
| 1861 | ttcgccatcc tgctgagtgt gggggcttgc atctccattg cactcaccta cttccagttg |
| 1921 | tctggggagg attaccgctg gtggtggcga tctgtgctga gtgttggctc caccggcctc |
| 1981 | ttcatcttcc tctactcagt tttctattat gcccggcgct ccaacatgtc tggggcagta |
| 2041 | cagacagtag agttcttcgg ctactcctta ctcactggtt atgtcttctt cctcatgctg |
| 2101 | ggcaccatct cctttttttc ttccctaaag ttcatccggt atatctatgt taacctcaag |
| 2161 | atggactgag ttctgtatgg cagaactatt gctgttctct ccctttcttc atgccctgtt |
| 2221 | gaactctcct accagcttct cttctgattg actgaattgt gtgatggcat tgttgccttc |
| 2281 | ccttttgccc tttgggcatt ccttccccag agagggcctg gaaattataa atctctatca |
| 2341 | cataaggatt atatatttga actttttaag ttgcctttag ttttggtcct gatttttctt |
| 2401 | tttacaatta ccaaaataaa atttattaag aaaaaggaaa aaaaaaaaa |

Transmembrane 9 superfamily member 1 isoform b (Homo sapiens)
VERSION NP_001014842.1 GI:62460635
DEBOURCE REFSEQ: accession NM_001014842.1
(SEQ ID NO: 3)

| | |
|---|---|
| 1 | ggccgcgctg ccgatcgccg ggaggacccc cgcctcgccg aagacgggcg gggcaagccg |
| 61 | agcctcacgg ggtccccgga gctgggccgg gcctccagat ggagaaggcg caacggggag |
| 121 | ttcttgagta agccagagcg gtgtccagcg cggtgtagcc gcagccgccg ctgtcaggcg |
| 181 | cagcaacggg caaccccgta gaagtcggtc ggcaggtcct ctccaacccg ccgctaccgc |
| 241 | gccgctgtgg gagagacccc agcaggagcc caaaggcagc tacgggggcg cgaaggccgc |
| 301 | tggcgccgcc tcggccagcc cttcccgcgc ggttccactg ccttaaggat gacagtcgta |
| 361 | gggaaccctc gaagttggag ctgccagtgg ttgccaatcc tgatactgtt gctgggcaca |
| 421 | ggccatgggc caggggtgga aggcgtgaca cactacaagg ccggcgaccc tgttattctg |
| 481 | tatgtcaaca aagtgggacc ctaccataac cctcaggaaa cttaccacta ctatcagctt |
| 541 | ccagtctgct gccctgagaa gatacgtcac aaaagcctta gcctgggtga agtgctggat |
| 601 | ggggaccgaa tggctgagtc tttgtatgag atccgctttc gggaaaacgt ggagaagaga |
| 661 | attctgtgcc acatgcagct cagttctgca caggtggagc agctgcgcca ggccattgaa |
| 721 | gaactgtact actttgaatt tgtggtagat gacttgccaa tccggggctt tgtgggctac |
| 781 | atggaggaga gtggtttcct gccacacagc cacaagatag gactctggac ccatttggac |
| 841 | ttccacctag aattccatgg agaccgaatt atatttgcca atgtttcagt gcgggacgtc |
| 901 | aagccccaca gcttggatgg gttacgacct gacgagttcc taggccttac ccacacttat |
| 961 | agcgtgcgct ggtctgagac ttcagtggag cgtcggagtg acaggcgccg tggtgacgat |
| 1021 | ggtggtttct ttcctcgaac actggaaatc cattggttgt ccatcatcaa ctccatggtg |
| 1081 | cttgtgtttt tactggtggg ttttgtggct gtcattctaa tgcgtgtgct tcggaatgac |
| 1141 | ctggctcggt acaacttaga tgaggagacc acctctgcag gttctggtga tgactttgac |
| 1201 | cagggtgaca atggctggaa aattatccat acagatgtct tccgcttccc cccataccgt |
| 1261 | ggtctgctct gtgctgtgct tggcgtgggt gcccagttcc tggcccttgg cactggcatt |
| 1321 | attgtcatgg cactgctggg catgttcaat gtgcaccgtc atggggccat taactcagca |
| 1381 | gccatcttgt tgtatgccct gacctgctgc atctctggct acgtgtccag ccacttctac |
| 1441 | cggcagattg gaggcgagcg ttgggtgtgg aacatcattc tcaccaccag tctcttctct |
| 1501 | gtgcctttct tcctgacgtg gagtgtggtg aactcagtgc attgggccaa tggttcgaca |
| 1561 | caggctctgc cagccacaac catcctgctg cttctgacgg tttggctgct ggtgggcttt |
| 1621 | cccctcactg tcattggagg catctttggg aagaacaacg ccagcccctt tgatgcaccc |
| 1681 | tgtcgcacca agaacatcgc ccgggagatt ccaccccagc cctggtacaa gtctactgtc |
| 1741 | atccacatga ctgttggagg cttcctgcct ttcaggtatc ctccctttat tccatggcta |
| 1801 | ttactgtcag gttcctgacc tcaatttttc ctgtccctac tcatccagta ccctaaccca |
| 1861 | acccgttgat ccctggttca gtggtaccat tcagagatca ttaaatggtt cctcctatcc |
| 1921 | ccaagcagga ctgagcttga atgatatgag agtgtctcac ttataaagct ctccggagac |
| 1981 | atttccccct tcaccttcct ggtttctgac tttaatgcct atggacatca tgtggggttt |
| 2041 | aaagcccatt tgatgaccca tttactttgt tgaatacctc tttgtgccag gcaaagaata |
| 2101 | aagtggaata aaatggaaaa aaaa |

Transmembrane 9 superfamily member 2 (Homo sapiens)
VERSION NP_004791.1 GI:4758874
DBSOURCE REFSEQ: accession NM_004800.1
(SEQ ID NO: 5)

| | |
|---|---|
| 1 | cgcaaccgga actagccttc tgggggccgg cttggtttat ctctggcggc cttgtagtcg |
| 61 | tctccgagac tccccacccc tccttccctc ttgaccccct aggtttgatt gccctttccc |
| 121 | cgaaacaact atcatgagcg cgaggctgcc ggtgttgtct ccacctcggt ggccgcggct |
| 181 | gttgctgctg tcgctgctcc tgctgggggc ggttcctggc ccgcgccgga gcggcgcttt |
| 241 | ctacctgccc ggcctggcgc ccgtcaactt ctgcgacgaa gaaaaaaaga gcgacgagtg |
| 301 | caaggccgaa atagaactat ttgtgaacag acttgattca gtggaatcag ttcttcctta |
| 361 | tgaatacaca gcgtttgatt tttgccaagc atcagaagga aagcgcccat ctgaaaatct |
| 421 | tggtcaggta ctattcgggg aaagaattga accttcacca tataagttta cgtttaataa |
| 481 | gaaggagacc tgtaagcttg tttgtacaaa aacataccat acagagaaag ctgaagacaa |
| 541 | acaaaagtta gaattcttga aaaaaagcat gttattgaat tatcaacatc actggattgt |
| 601 | ggataatatg cctgtaacgt ggtgttacga tgttgaagat ggtcagaggt tctgtaatcc |
| 661 | tggatttcct attggctgtt acattacaga taaaggccat gcaaaagatg cctgtgttat |
| 721 | tagttcagat ttccatgaaa gagatacatt ttacatcttc aaccatgttg acatcaaaat |
| 781 | atactatcat gttgttgaaa ctgggtccat gggagcaaga ttagtggctg ctaaacttga |
| 841 | accgaaaagc ttcaaacata cccatataga taaaccagac tgctcagggc cccccatgga |
| 901 | cataagtaac aaggcttctg gggagataaa aattgcctat acttactctg ttagcttcga |
| 961 | ggaagatgat aagatcagat gggcgtctag atgggactat attctggagt ctatgcctca |
| 1021 | tacccacatt cagtggttta gcattatgaa ttccctggtc attgttctct tcttatctgg |
| 1081 | aatggtagct atgattatgt tacggacact gcacaaagat attgctagat ataatcagat |
| 1141 | ggactctacg gaagatgccc aggaagaatt tggctggaaa cttgttcatg gtgatatatt |
| 1201 | ccgtcctcca agaaaaggga tgctgctatc agtctttcta ggatccggga cacagatttt |
| 1261 | aattatgacc tttgtgactc tatttttcgc ttgcctggga tttttgtcac ctgccaaccg |
| 1321 | aggagcgctg atgacgtgtg ctgtggtcct gtgggtgctg ctgggcaccc ctgcaggcta |
| 1381 | tgttgctgcc agattctata agtcctttgg aggtgagaag tggaaaacaa atgttttatt |
| 1441 | aacatcattt ctttgtcctg ggattgtatt tgctgacttc tttataatga atctgatcct |
| 1501 | ctggggagaa ggatcttcag cagctattcc ttttgggaca ctggttgcca tattggccct |
| 1561 | ttggttctgc atatctgtgc ctctgacgtt tattggtgca tactttggtt ttaagaagaa |
| 1621 | tgccattgaa cacccagttc gaaccaatca gattccacgt cagattcctg aacagtcgtt |
| 1681 | ctacacgaag cccttgcctg gtattatcat gggagggatt ttgccctttg gctgcatctt |
| 1741 | tatacaactt ttcttcattc tgaatagtat ttggtcacac cagatgtatt acatgtttgg |
| 1801 | cttcctattt ctggtgttta tcattttggt tattacctgt tctgaagcaa ctatacttct |
| 1861 | ttgctatttc cacctatgtg cagaggatta tcattggcaa tggcgttcat tccttacgag |
| 1921 | tggctttact gcagtttatt tcttaatcta tgcagtacac tacttctttt caaaactgca |
| 1981 | gatcacggga acagcaagca caattctgta ctttggttat accatgataa tggttttgat |
| 2041 | cttctttctt tttacaggaa caattggctt ctttgcatgc ttttggtttg ttaccaaaat |
| 2101 | atacagtgtg gtgaaggttg actgaagaag tccagtgtgt ccagttaaaa cagaaataaa |
| 2161 | ttaaactctt catcaacaaa gacctgtttt tgtgactgcc ttgagtttta tcagaattat |
| 2221 | tggcctagta atccttcaga aacaccgtaa ttctaaataa acctcttccc atacaccttt |
| 2281 | cccccataag atctgtcttc aacactataa agcatttgta ttgtgatttg attaagtata |
| 2341 | tatttggttg ttctcaatga agagcaaatt taaatattat gtgcatttga a |

LOCUS NP_064508 589 aA linear PRI 12 Jun. 2008
Transmembrane 9 superfamily member 3 (Homo sapiens)
VERSION NP_064508.3 GI:190194386
DBSOURCE REFSEQ: accession NM_020123.3
(SEQ ID NO: 7)

| | |
|---|---|
| 1 | gaggaagagg ctgaggaggc gcggggggcg ggggaggctc aggagcgggc ggtgacggcg |
| 61 | acggcggcgg cagaggaggc agcggctggg ccgggccccg tgcgtctgtc cgcgccccgt |
| 121 | ggatgcgaat cggccgcggc ggaggcggcg gcggcggagg aggcggcggc gggaggagga |
| 181 | gtcggtgagc cggctccggg ccggaggggc gcggaggatg aggccgctgc ctggcgctct |
| 241 | tggcgtggcg gcggccgccg cgctgtggct gctgctgctg ctgctgcccc ggacccgggc |
| 301 | ggacgagcac gaacacacgt atcaagataa agaggaagtt gtcttatgga tgaatactgt |
| 361 | tgggccctac cataatcgtc aagaaacata taagtacttt tcacttccat tctgtgtggg |
| 421 | gtcaaaaaaa agtatcagtc attaccatga aactctggga gaagcacttc aaggggttga |
| 481 | attggaattt agtggtctgg atattaaatt taaagatgat gtgatgccag ccacttactg |
| 541 | tgaaattgat ttagataaag aaaagagaga tgcatttgta tatgccataa aaaatcatta |
| 601 | ctggtaccag atgtacatag atgatttacc aatatggggt attgttggtg aggctgatga |
| 661 | aaatggagaa gattactatc tttggaccta taaaaaactt gaaataggtt ttaatggaaa |
| 721 | tcgaattgtt gatgttaatc taactagtga aggaaaggtg aaactggttc caaatactaa |
| 781 | aatccagatg tcatattcag taaaatggaa aaagtcagat gtgaaatttg aagatcgatt |
| 841 | tgacaaatat cttgatccgt ccttttttca acatcggatt cattggtttt caattttcaa |
| 901 | ctccttcatg atggtgatct tcttggtggg cttagtttca atgattttaa tgagaacatt |
| 961 | aagaaaagat tatgctcggt acagtaaaga ggaagaaatg gatgatatgg atagagacct |
| 1021 | aggagatgaa tatggatgga aacaggtgca tggagatgta tttagaccat caagtcaccc |
| 1081 | actgatattt tcctctctga ttggttctgg atgtcagata tttgctgtgt ctctcatcgt |
| 1141 | tattattgtt gcaatgatag aagatttata tactgagagg ggatcaatgc tcagtacagc |
| 1201 | catatttgtc tatgctgcta cgtctccagt gaatggttat tttggaggaa gtctgtatgc |
| 1261 | tagacaagga ggaaggagat ggataaagca gatgtttatt ggggcattcc ttatcccagc |
| 1321 | tatggtgtgt ggcactgcct tcttcatcaa tttcatagcc atttattacc atgcttcaag |
| 1381 | agccattcct tttggaacaa tggtggccgt ttgttgcatc tgtttttttg ttattcttcc |
| 1441 | tctaaatctt gttggtacaa tacttggccg aaatctgtca ggtcagccca actttccttg |
| 1501 | tcgtgtcaat gctgtgcctc gtcctatacc ggagaaaaaa tggttcatgg agcctgcggt |
| 1561 | tattgtttgc ctgggtggaa ttttaccttt tggttcaatc tttattgaaa tgtatttcat |
| 1621 | cttcacgtct ttctgggcat ataagatcta ttatgtctat ggcttcatga tgctggtgct |
| 1681 | ggttatcctg tgcattgtga ctgtctgtgt gactattgtg tgcacatatt ttctactaaa |
| 1741 | tgcagaagat taccggtggc aatggacaag ttttctctct gctgcatcaa ctgcaatcta |
| 1801 | tgtttacatg tattcctttt actactattt tttcaaaaca aagatgtatg gcttatttca |
| 1861 | aacatcattt tactttggat atatggcggt atttagcaca gccttgggga taatgtgtgg |
| 1921 | agcgattggt tacatgggaa caagtgcctt tgtccgaaaa atctatacta atgtgaaaat |
| 1981 | tgactagaga cccaagaaaa cctggaactt tggatcaatt tctttttcat aggggtggaa |
| 2041 | cttgcacagc aaaaacaaac aaacgcaaga agagatttgg gctttaacac actgggtact |
| 2101 | ttgtgggtct ctctttcgtc ggtggcttaa agtaacatct atttccattg atcctaggtt |
| 2161 | cttcctgact gctttctcca actgttcaca gcaaatgctt ggattttatg cagtaggcat |
| 2221 | tactacagta catggctaat cttcccaaaa actagctcat taaagatgaa atagaccagc |
| 2281 | tctcttcagt gaagaggaca aatagtttat ttaaagcatt tgttccaata aaataaatag |
| 2341 | agggaaactt ggatgctaaa attacatgaa taggaatctt cctggcactt agtgtttcta |
| 2401 | tgttattgaa aaatgatgtt ccagaaagat tacttttttc ctcttatttt tactgccatt |
| 2461 | gtcgacctat tgtgggacat ttttatatat tgaatctggg ttcttttttg actttttttt |
| 2521 | tttcccaatc caacagcatc ctttttttta aaagagagaa ttagaaaata ttaaatcctg |
| 2581 | catgtaatat atctgctgtc atcttagttg gaccaacttc ccatttattt atcttaaaac |
| 2641 | tatacagtta catcttaatt ccatccaaag aagatacagt ttgaagacag aagtgtactc |
| 2701 | tctacaatgc aatttactgt acagttagaa agcaaagtgt taaatggaga agatacttgt |
| 2761 | ttttattaaa cattttgaga tttagataaa ctacatttta actgaatgtc taaagtgatt |
| 2821 | atcttttttc cccccaagtt agtcttaaat cttttgggtt tgaatgaagg ttttacataa |
| 2881 | gaaattatta aaaacaaggg gggtgggtaa taaatgtata taacattaaa taatgtaacg |
| 2941 | taggtgtaga ttcccaaatg catttggatg tacagatcga ctacagagta cttttttctt |
| 3001 | atgatgattg gtgtagaaat gtgtgatttg ggtgggcttt tacatcttgc ctaccattgc |
| 3061 | atgaaacatt ggggtttctt caaaatgtgt gtgtcatact tcttttggga ggggggttgt |
| 3121 | tttcttctgt ttattttctg agactcctac aggagccaaa tttgtaattt agagacactt |
| 3181 | aattttgtta atcctgtctg ggacacttaa gtaacatcta aagcattatt gctttagaat |
| 3241 | gttcaaataa aatttcctga ccaaattgtt ttgtggaaat agatgtgttt gcaatttgaa |
| 3301 | gatatctttc tgtccagaag gcaaaattac cgaatgccat ttttaaaagt atgctataaa |
| 3361 | ctatgctact ctcatacagg ggacccgtat tttaaaatct ccagacttgc ttacatctag |
| 3421 | attatccagc acaatcataa agtgaatgac aaaccctttg aatgaaattg tggcacaaaa |
| 3481 | tctgttcagg ttggtgtacc gtgtaaagtg gggatggggt aaaagtggtt aacgtactgt |
| 3541 | tggatcaaca aataaaggtt acagttttgt aagagaagtg atttgaatac atttttctgg |
| 3601 | aactattcat aatatgaagt tttcctagaa ccactgagtt tctagtttaa tagtttgcta |
| 3661 | tgcaaatgac cacctaaaac aatactttat attgttattt ttagaaagac tcaaaacacc |
| 3721 | tgtatttaaa ccttaatatg aaaatcatgc aattaatagt tacacaagat gttttcatta |
| 3781 | caaaatatgt acctatctat tgatggactc tacatcctat attgtgacat gtaagtcctt |
| 3841 | taaaaggtga aaagtatgat ttcttaccac ttaagtatga ttgatatgat ccaacaaatt |
| 3901 | tgatcagaag ctgtaggtaa atcctcttct gaagccaaaa tggtatatta aatataattt |
| 3961 | attggtactt ccattttctc ttccttctta cttgccttta agatcttata aaaaagaaac |
| 4021 | taaaagttaa tatttagttg cctatattat gtaacctttt aactatatat aaagtacttt |
| 4081 | tttggtttct ttctcaccac ttttattcaa aagtactttt aacataccaa tacatagtct |
| 4141 | gtctgatggg agtataaatt ggacagtaag gttttgtctt aataaaatga aatttgtttc |
| 4201 | tcatgatatg aatcttgcag gtaagatgta gggtttattg aaaatgtgtg ggttaaatgc |
| 4261 | tttcaggtac accaattctt tctactaaat tgagctctat ttgaagttct ttggaatctg |
| 4321 | tggtgaaaaa taattttctg atttccaaat acattaagag cattaaatga atattaatca |
| 4381 | cctttaaagt cttttagaaa aggacttgta ttggtttttg gctgcataga ggggttgaat |
| 4441 | aagtgtatgt atgtgtgtgc gtgtgtgtgt gtcttcttaa agaagatgta attcacaaat |
| 4501 | agtttagctc cctagcgctc agttgtagaa tagaaaatag aacattattc aagttaattg |
| 4561 | aaaggtgagg tttttatacc cccactaatg ctgtgtatct gtctttcgtt tgttaacatt |
| 4621 | atttgcttaa tttctttcaa ctcacacttt ggataatact atcaaaaact aaggctaaac |
| 4681 | attccttgtg tatctttaag catgcttctc ctgaaattta actacattag tagttgacat |
| 4741 | ttgtatacat atatcctaat acaagagtag gataaggtgg aaatgtaatg gcctgaggga |
| 4801 | tggtgaagca ttcttttagt atttttcatc atgttgggct cctagattgt actggggttg |
| 4861 | cccataaatc aaaccccata ctcttagaat tcattatatt atggtgatat ccgaacctag |
| 4921 | tgaatggtat gcttgggtgt tttccattga gagtggatgg acctctttat aaagttggtt |
| 4981 | gctgcaaaat ccagttcttc caaaagccac tttatttagg gtttattcac aagtcatatc |
| 5041 | cattttggta cagtgtttgt ttcctaatat ttattaacca ccttatacca aatgtcttgc |
| 5101 | aaagaaatgt tattaaaacc ttgaattttt acaaatgtaa aaaacaaaaa gtgtattaat |
| 5161 | gtatttgttc aggaaaagct acataccgaa gggcttttgt atatgaattc tgtggtgggg |
| 5221 | agacccattt gtaatctata tggcagttcc atctgggttt taagtttaga tttcaccgtg |
| 5281 | tcttagtgct tcattctatt ggtttattgg aacatgtaat aaataggagt agtgatgtat |
| 5341 | taaaacacaa gtattcatta atgttttata tcttcactaa aattctatag ttatgaaact |
| 5401 | atcaatcaag gtgttatatt tcagtcagaa gtgaaaattt atgaagagta tttggaagtg |
| 5461 | tgtacagaaa taaactagac ttacaggtag gctagatcag aacgttaaca tatgaacctg |
| 5521 | cagaaatctg gtaagactta aattcagtgt gaggaataac tctagttctc tcctatgagc |
| 5581 | atttcctaaa agccatctga tttggcattc ttactggagc tgcagacaga aatctacaaa |
| 5641 | gacaaaagta aacaaaatta agttattatt ccactgttag gaatggaaat aaacttgtga |
| 5701 | agtctgttta ttttgaagta ttggtgaact aggcttgcta attgataact gcagcagttt |
| 5761 | gtgtttactc cagttcatca gcttaggtca tttgaaagat ataagagctt aaggcaagaa |
| 5821 | agaaataaca tggaattcta tttgaaggac aacagaacat tcttggaaaa gcagctccag |
| 5881 | ttggtttttc aactgtcaaa cttgaatgtg taagtcccca cagagcatgg acagtcggtg |
| 5941 | cagagttcca aggaaacaat tattgcctga tgaccacttc cattttgtat acactctttg |
| 6001 | gttcgtatag gccatattcc aactggcttt ttagtaatag aaatccagta tataatgtat |
| 6061 | caaatacaat tgaggttcta acctagtgtg ttaatttatc tgaatttgga tttttaaaaa |
| 6121 | gtaataaaaa gttaaatgta |

Transmembrane 9 superfamily protein member 4 (Homo sapiens)
VERSION NP_055557.2 GI:164519076
DB SOURCE REFSEQ: accession NM_014742.3
(SEQ ID NO: 9)

| | |
|---|---|
| 1 | agtttctgcc aggagctaat atggcttcct tagttacacc gttctctctc ttcacctaat |
| 61 | cagcgacctt actttcccag accagactgt cgagcaggag ctaagactcc ttttcccctc |
| 121 | tgctgaccgc cactacagga gcggttgaag ccagacgacc accttgtgga gttaaactcc |
| 181 | gtaaccaggg agcaccactt ccgctgacgt cattacggcg acacgtggat ccaagatggc |
| 241 | gacggcgatg gattggttgc cgtggtcttt actgcttttc tccctgatgt gtgaaacaag |
| 301 | cgccttctat gtgcctgggg tcgcgcctat caacttccac cagaacgatc ccgtagaaat |
| 361 | caaggctgtg aagctcacca gctctcgaac ccagctacct tatgaatact attcactgcc |
| 421 | cttctgccag cccagcaaga taacctacaa ggcagagaat ctgggagagg tgctgagagg |
| 481 | ggaccggatt gtcaacaccc ctttccaggt tctcatgaac agcgagaaga agtgtgaagt |
| 541 | tctgtgcagc cagtccaaca agccagtgac cctgacagtg gagcagagcc gactcgtggc |
| 601 | cgagcggatc acagaagact actacgtcca cctcattgct gacaacctgc ctgtggccac |
| 661 | ccggctggag ctctactcca accgagacag cgatgacaag aagaaggaaa aagatgtgca |
| 721 | gtttgaacac ggctaccggc tcggcttcac agatgtcaac aagatctacc tgcacaacca |
| 781 | cctctcattc atcctttact atcatcggga ggacatggaa gaggaccagg agcacacgta |
| 841 | ccgtgtcgtc cgcttcgagg tgattcccca gagcatcagg ctggaggacc tcaaagcaga |
| 901 | tgagaagagt tcgtgcactc tgcctgaggg taccaactcc tcgccccaag aaattgaccc |
| 961 | caccaaggag aatcagctgt acttcaccta ctctgtccac tgggaggaaa gtgatatcaa |
| 1021 | atgggcctct cgctgggaca cttacctgac catgagtgac gtccagatcc actggttttc |
| 1081 | tatcattaac tccgttgttg tggtcttctt cctgtcaggt atcctgagca tgattatcat |
| 1141 | tcggaccctc cggaaggaca ttgccaacta caacaaggag gatgacattg aagacaccat |
| 1201 | ggaggagtct gggtggaagt tggtgcacgg cgacgtcttc aggccccccc agtaccccat |
| 1261 | gatcctcagc tccctgctgg gctcaggcat tcagctgttc tgtatgatcc tcatcgtcat |
| 1321 | ctttgtagcc atgcttggga tgctgtcgcc ctccagccgg ggagctctca tgaccacagc |
| 1381 | ctgcttcctc ttcatgttca tgggggtgtt tggcggattt tctgctggcc gtctgtaccg |
| 1441 | cactttaaaa ggccatcggt ggaagaaagg agccttctgt acggcaactc tgtaccctgg |
| 1501 | tgtggttttt ggcatctgct tcgtattgaa ttgcttcatt tggggaaagc actcatcagg |
| 1561 | agcggtgccc tttcccacca tggtggctct gctgtgcatg tggttcggga tctccctgcc |
| 1621 | cctcgtctac ttgggctact acttcggctt ccgaaagcag ccatatgaca accctgtgcg |
| 1681 | caccaaccag attccccggc agatccccga gcagcggtgg tacatgaacc gatttgtggg |
| 1741 | catcctcatg gctgggatct tgcccttcgg cgccatgttc atcgagctct tcttcatctt |
| 1801 | cagtgctatc tgggagaatc agttctatta cctctttggc ttcctgttcc ttgttttcat |
| 1861 | catcctggtg gtatcctgtt cacaaatcag catcgtcatg gtgtacttcc agctgtgtgc |
| 1921 | agaggattac cgctggtggt ggagaaattt cctagtctcc gggggctctg cattctacgt |
| 1981 | cctggtttat gccatctttt atttcgttaa caagctggac atcgtggagt tcatcccctc |
| 2041 | tctcctctac tttggctaca cggccctcat ggtcttgtcc ttctggctgc taacgggtac |
| 2101 | catcggcttc tatgcagcct acatgtttgt tcgcaagatc tatgctgctg tgaagataga |
| 2161 | ctgattggag tggaccacgg ccaagcttgc tccgtcctcg gacaggaagc caccctgcgt |
| 2221 | gggggactgc aggcacgcaa aataaaataa ctcctgctcg tttggaatgt aactcctggc |
| 2281 | acagtgttcc tggatcctgg ggctgcgtgg ggggcgggag ggcctgtaga taatcttgcg |
| 2341 | tttttcgtca tcttattcca gttctgtggg ggatgagttt ttttgtgggt tgctttttct |
| 2401 | tcagtgctaa gaaagttccc tccaacagga actctctgac ctgtttattc aggtgtattt |
| 2461 | ctggtttgga tttttttttc cttctttgtt ttaacaaatg gatccaggat ggataaatcc |
| 2521 | accgagataa gggttttggt cactgtctcc acctcagttc ctcagggctg ttggccaccc |
| 2581 | tatgactaac tggaagagga cacgccagag cttcagtgag gtttccgagc ctctccctgc |
| 2641 | ccatcctcac cactgaggcc acgacaaagc acagctccag ctcggacagc accctcagtg |
| 2701 | ccagccagcc tctgccagac ctctctttcc ctcttctccc cagcctcctc cagggctgcc |
| 2761 | caaggcaggg tttccagcca ggcctcgggg tcatcttttc accaggagca aacccaagtc |
| 2821 | ttagttgcta caagaaaatc ccctggaagt actgggggcc aggttcccca gacagcagga |
| 2881 | attgcccctg ttcagagcag ccggagtttg ctggaccaca aggaagaaga gaagagactt |
| 2941 | gcagtgaact gtttttgtgc caagaaaccc tggacctggg gccaagtatt tcccaagcca |
| 3001 | agcatccact tgtctgtgtc tgggaaggga tggccaaggc cgctagggtc cttacccctc |
| 3061 | aggatcactc cccagccctt tcctcaggag gtaccgctct ccaaggtgtg ctagcagtgg |
| 3121 | gccctgccca acttcaggca gaacagggag gcccagagat tacagatccc ctcctgtaag |
| 3181 | tggccaggca ttctctccct gccctctctg gcctctgggg tcatactcac ttctttagcc |
| 3241 | agccccatcc cctccacccc acacctgagt tcttgcctcc tccttttggg gacacccaaa |
| 3301 | acactgcttg tgagaaggaa gatggaaggt aagttctgtc gttctttccc caatccccag |
| 3361 | gaatggacaa gaagccaact tagaaagaag ggtctcacgt ggctggcctg gctcctccgt |
| 3421 | agacccctgt tcttttcaac ctctgcccac ccgtgcatgt catcacaaac atttgctctt |
| 3481 | aagttacaag agaccacatc cacccaggga ttagggttca agtagcagct gctaaccctt |
| 3541 | gcaccagccc ttgtgggact cccaacacaa gacaaagctc aggatgctgg tgatgctagg |
| 3601 | aagatgtccc tcccctcact gccccacatt ctcccagtgg ctctaccagc ctcacccatc |
| 3661 | aaaccagtga atttctcaat cttgcctcac agtgactgca gcgccaagcg gcatccacca |
| 3721 | agcatcaagt tggagaaaag ggaacccaag cagtagagag cgatattgga gtcttttgtt |
| 3781 | cattcaaatc ttggattttt ttttttccct aagagattct ctttttaggg ggaatgggaa |
| 3841 | acggacacct cataaagggt tcaaagatca tcaatttttc tgacttttta aatcattatc |
| 3901 | attattattt ttaattaaaa aaatgcctgt atgccttttt ttggtcggat tgtaaataaa |
| 3961 | tataccattg tcctactgaa aaaaaaaaaa aaaaaa |

TABLE 2
Protein sequences of arNOX transmembrane 9 superfamily
proteins 1a, 1b, 2, 3 and 4 (Homo sapiens)

Transmembrane 9 superfamily member 1 isoform a
(SEQ ID NO: 2)
MTVVGNPRSWSCQWLPILILLLGTGHGPGVEGVTHYKAGDPVILYVNKVGPYHNPQETYHYYQLPVCCPEKTR
HKSLSLGEVLDGDRMAESLYEIRFRENVEKRILCHMQLSSAQVEQLRQAIEELYYFEFVVDDLPIRGFVGYME
ESGFLPHSHKIGLWTHLDFHLEFHGDRIIFANVSVRDVKPHSLDGLRPDEFLGLTHTYSVRWSETSVERRSDR
RRGDDGGFFPRTLEIHWLSIINSMVLVFLLVGFVAVILMRVLRNDLARYNLDEETTSAGSGDDFDQGDNGWKI
IHTDVERFPPYRGLLCAVLGVGAQFLALGTGIIVMALLGMFNVHRHGAINSAAILLYALTCCISGYVSSHFYR
QIGGFRWVWNIILTTSLFSVPFFLTWSVVNSVHWANGSTQALPATTILLLLTVWLLVGFPLTVIGGIFGKNNA
SPFDAPCRTKNIAREIPPQPWYKSTVIHMTVGGFLPFSAISVELYYIFATVWGREQYTLYGILFFVFAILLSV
GACISIALTYFQLSGEDYRWWWRSVLSVGSTGLFIFLYSVFYYARRSNM
SGAVQTVEFFGYSLLTGYVFFLMLGTISFFSSLKFIRYIYVNLKMD
Transmembrane 9 superfamily member 1 isoform b
(SEQ ID NO: 4)
MTVVGNPRSWSCQWLPILILLLGTGHGPGVEGVTHYKAGDPVILYVNKVGPYHNPQETYHYYQLPVCCPEKIR
HKSLSLGEVLDGDRMAESLYEIRFRENVEKRILCHMQLSSAQVEQLRQAIEELYYFEEVVDDLPIRGFVGYME
ESGFLPHSHKIGLWTHLDFHLEFHGDRIIFANVSVRDVKPHSLDGLRPDEFLGLTHTYSVRWSETSVERRSDR
RRGDDGGFFPRTLEIHWLSIINSMVLVFLLVGFVAVILMRVLRNDLARYNLDEETTSAGSGDDFDQGDNGWKI
IHTDVFRFPPYRGLLCAVLGVGAQFLALGTGIIVMALLGMFNVHRHGAINSAAILLYALTCCISGYVSSHFYR
QIGGERWVWNIILTTSLFSVPFFLTWSVVNSVHWANGSTQALPATTILLLLTVWLLVGFPLTVIGGIFGKNNA
SPFDAPCRTKNIAREIPPQPWYKSTVIHMTVGGFLPFRYPPFIPWLLLSGS
Transmembrane 9 superfamily member 2
(SEQ ID NO: 6)
MSARLPVLSPPRWPRLLLLSLLLLGAVPGPRRSGAFYLPGLAPVNFCDEEKKSDECKAEIELFVNRLDSV
ESVLPYEYTAFDFCQASEGKRPSENLGQVLFGERIEPSPYKFTFNKKETCKLVCTKTYHTEKAEDKQKLE
FLKKSMLLNYQHHWIVDNMPVTWCYDVEDGQRFCNPGFPIGCYITDKGHAKDACVISSDFHERDTFYIRN
HVDIKIYYHVVE′TGSMGARLVAAKLEPKSFKHTHIDKPDCSGPPMDISNKASGEIKIAYTYSVSFEEDDKIRWAS
RWDYILESMPHTHIQWFSIMNSLVIVLFLSGMVAMIMLRTLHKDIARYNQMDSTEDAQEEFGWKLVHGDIFRPPRK
GMLLSVFLGSGTQILIMTFVTLFFACLGFLSPANRGALMTCAVVLWVLLGTPAGYVAARTYKSFGGEKWKTNVLLT
SFLCPGIVFADFFIMNLILWGEGSSAAIPFGTLVAILALWFCISVPLTFIGAYFGFKKNAIEHPVRTNQIPRQIPE
QSFYTKPLPGIIMGGILPFGCIFIQLFFILNSIWSHQMYYMFGFLFLVFIILVITCSEATILLCYFHLCAEDYHWQ
WRSFLTSGFTAVYFLIYAVHYFFSKLQITGTASTILYFGYT MIMVLIEFLFTGTIGFFACFWFVTKIYSVVKVD
Transmembrane 9 superfamily member 3
(SEQ ID NO: 8)
MRPLPGALGVAAAAALWLLLLLLPRTRADEHEHTYQDKEEVVLWMNTVGPYHNRQETYKYFSLPFCVGSK
KSISITYHETLGEALQGVELEFSGLDIKFKDDVMPATYCEIDLDKEKRDAFVYAIKNHYWYQMYIDDLPIW
GIVGEADENGEDYYLWTYKKLEIGFNGNRIVDVNLTSEGKVKLVPNTKIQMSYSVKWKKSDVKFEDRFDK
YLDPSFFQHRIHNFSIFNSFMMVIFLVGLVSMILMRTLRKDYARYSKEEEMDDMDRDLGDEYGWKQVHGD
VFRPSSHPLIFSSLIGSGCQIFAVSLIVIIVAMIEDLYTERGSMLSTAIFVYAATSPVNGYFGGSLYARQ
GGRRWIKQMFIGAFLIPAMVCGTAFFINFIAIYYHASRAIPFGTMVAVCCICFFVILPLNLVGTILGRNL
SGQPNFPCRVNAVPRPIPEKKWFMFPAVIVCLGGILPFGSIFIEMYFIFTSFWAYKIYYVYGFMMLVLVI
LCIVTVCVTIVCTYFLLNAEDYRWQWTSFLSAASTAIYVYMYSFYYYFFKTKMYGLFQTSFYFGYMAVFS
TALGIMCGAIGYMGTSAFVRKIYTNVKID
Transmembrane 9 superfamily member 4
(SEQ ID NO: 10)
MATAMDWLPWSLLLFSLMCETSAFYVPGVAPINFHQNDPVEIKAVKLTSSRTQLPYEYYSLPFCQPSKIT
YKAENLGEVLRGDRIVNTPFQVLMNSEKKCEVLCSQSNKPVTLTVEQSRLVAERITEDYYVHLIADNLPV
ATRLELYSNRDSDDKKKEKDVQFEHGYRLGFTDVNKIYLHNHLSFILYYHREDMEEDQEHTYRVVRFEVI
PQSIRLEDLEADEKSSCTLPEGTNSSPQEIDPTKENQLYFTYSVHWEESDIKWASRWDTYLTMSDVQIHW
FSIINSVVVVFFLSGILSMIIIRTLRKDIANYNKEDDIEDTMEESGWKLVHGDVFRPPQYPMILSSLLGS
GIQLFCMILIVIFVAMLGMLSPSSRGALMTTACFLFMFMGVFGGFSAGRLYRTLKGHRWKKGAFCTATLY
PGVVFGICFVLNCFIWGKHSSGAVPFPTMVALLCMWFGISLPLVYLGYYFGFRKQPYDNPVRTNQIPRQI.
PEQRWYMNRFVGILMAGILPFGAMFIELFFIFSAIWENQFYYLFGFLFLVFIILVVSCSQISIVMVYFQL
CAEDYRWWWRNFLVSGGSAFYVLVYAIFYFVNKLDIVEFIPSLLYFGYTALMVLSFWLLTGTIGFYAAYM
FVRKIYAAVKID

TABLE 3
Amino Acid Sequences of Human Soluble arNOX Enzymes

Transmembrane 9 superfamily member 1a (Homo sapiens)
(SEQ ID NO: 13)

| | |
|---|---|
| 1 | MTVVGNPRSW SCQWLPILIL LLGTGHGPGV EGVTHYKAGD PVILYVNKVG PYHNPQETYH |
| 61 | YYQLPVCCPE KIRHKSLSLG EVLDGDRMAE SLYEIRFREN VEKRILCHMQ LSSAQVEQLR |
| 121 | QAIEELYYFE FVVDDLPIRG FVGYMEESGF LPHSHKIGLW THLDFHLEFH GDRIIFANVS |
| 181 | VRDVKPHSLD GLRPDEFLGL THTYSVRWSE TSVERRSDRR RGDDGGFFPR TLEIHWL |

Conserved CQ/CE
Adenine nucleotide binding site GXGXXG at amino acids 27-32
Putative protein disulfide interchange site CXXXL
Putative copper sites HYY and HSH
Transmembrane 9 superfamily member 1b (Homo sapiens)
(SEQ ID NO: 14)

| | |
|---|---|
| 1 | MTVVGNPRSW SCQWLPILIL LLGTGHGPGV EGVTHYKAGD PVILYVNKVG PYHNPQETYH |
| 61 | YYQLPVCCPE KIRHKSLSLG EVLDGDRMAE SLYEIRFREN VEKRILCHMQ LSSAQVEQLR |
| 121 | QAIEELYYFE FVVDDLPIRG FVGYMEESGF LPHSHKIGLW THLDFHLEFH GDRIIFANVS |
| 181 | VRDVKPHSLD GLRPDEFLGL THTYSVRWSE TSVERRSDRR RGDDGGFFPR TLEIHWL |

Conserved CQ/CE
Adenine nucleotide binding site GXGXXG at amino acids 27-32
Putative protein disulfide interchange site CXXXL
Putative copper sites HYY and HSH
Transmembrane 9 superfamily member 2 (Homo sapiens)
(SEQ ID NO: 15)

| | |
|---|---|
| 1 | MSARLPVLSP PRWPRLLLLS LLLLGAVPGP RRSGAFYLPG LAPVNFCDEE KKSDECKAEI |
| 61 | ELFVNRLDSV ESVLPYEYTA FDFCQASEGK RPSENLGQVL FGERIEPSPY KFTFNKKETC |
| 121 | KLVCTKTYHT EKAEDKQKLE FLKKSMLLNY QHHWIVDNMP VTWCYDVEDG QRFCNPGFPI |
| 181 | GCYITDKGHA KDACVISSDF HERDTFYIFN HVDIKIYYHV VETGSMGARL VAAKLEPKSF |
| 241 | KHTHIDKPDC |

Conserved CQ/CE
Adenine nucleotide binding site GXVXXG at amino acids 97-102
Putative protein disulfide interchange site CXXXC
Putative copper sites YQH and HTH
Transmembrane 9 superfamily member 3 (Homo sapiens)
(SEQ ID NO: 16)

| | |
|---|---|
| 1 | MRPLPGALGV AAAAALWLLL LLLPRTRADE HEHTYQDKEE VVLWMNTVGP YHNRQETYKY |
| 61 | FSLPFCVGSK KSISHYHETL GEALQGVELE FSGLDIKFKD DVMPATYCEI DLDKEKRDAF |
| 121 | VYAIKNHYWY QMYIDDLPIW GIVGEADENG EDYYLWTYKK LEIGFNGNRI VDVNLTSEGK |
| 181 | VKLVPNTKIQ MSYSVKWKES DVKFEDRFDK YLDPSFFQHR IHWFSIFNSF MMVIFLVGLV |

Putative copper sites HTY and HYH
Adenine nucleotide binding site GXAXXG at amino acids 81-86
Conserved CQ/CE and CV
Putative protein disulfide interchange site CXXXL
Transmembrane 9 superfamily member 4 (Homo sapiens)
(SEQ ID NO: 17)

| | |
|---|---|
| 1 | MATAMDWLPW SLLLFSLMCE TSAFYVPGVA PINFHQNDPV EIKAVKLTSS RTQLPYEYYS |
| 61 | LPFCQPSKIT YKAENLGEVL RGDRIVNTPF QVLMNSEKKC EVLCSQSNKP VTLTVEQSRL |
| 121 | VAERITEDYY VHLIADNLPV ATRLELYSNR DSDDKKKEKD VQFEHGYRLG FTDVNKIYLH |
| 181 | NHLSFILYYH REDMEEDQEH TYRVVRFEVI PQSIRLEDLK ADEKSSCTLP EGTNSSPQEI |
| 241 | DPTKENQLYF TY |

Conserved CQ/CE
Adenine nucleotide binding site GXVXXG (amino acids 77-82)
Putative protein disulfide interchange site CXXXC
Putative copper sites YVH and HGY

### Example 1

### Cloning and Expression of Soluble arNOX Protein Transmembrane 9 Superfamily (TM9SF) Isoform 4

pET11b vector and BL21 (DE3) competent cells were purchased from Novagen (Madison, Wis.). Plasmids carrying TM9SF4 sequence were prepared by inserting the soluble Tm9SF4 coding sequence into the pET11b vector (between NheI and BamHI sites). The TM9SF4 sequence was amplified from full length cDNA by PCR. The primers used are 5′-GATATACATATGGCTAGCATGGCGACGGCGATGGAT-3′ (forward) (SEQ ID NO:11) and 5′-TTGTTAGCAGCCGGATCCTCAGTCTATCTTCACAGC-3′ (reverse) (SEQ ID NO:12). The PCR products then were doubly digested with NheI and BamHI and were ligated to pET11B vector.

DNA sequences of the ligation products (pET11b-TM9SF4) were confirmed by DNA sequencing. Then pET11b-TM9SF4 was transformed to BL21 (DE3) competent cells. A single colony was picked and inoculated into the 5 ml LB+ampicillin (LB/AMP) medium. The overnight culture (1 ml) was diluted into 100 ml LB/AMP media (1:100 dilution). The cells were grown with vigorous shaking (250 rpm) at 37° C. to an OD₆₀₀ of 0.4-0.6 and IPTG (0.5 mM) was added for induction. Cultures were collected after 5 hr incubation with shaking (250 rpm) at 37° C. Expression of the soluble TM9SF4 of about 30 kDa was confirmed by SDS-PAGE with silver staining. Transformed cells were stored at −80° C. in a standard glycerol stock solution.

For expression of TM9SF4, a small amount of cells from an isolated colony grown on LB+Amp agar was inoculated into LB+Amp and grown for 8 hr and stored at 4° C. overnight. Then the culture was centrifuged at 6,000 rpm for 6 min. The supernatant was discarded, and the cell pellet was resuspended in 4 ml of LB+amp medium and inoculated 1:100 into LB/amp medium and grown for 8 hr. No IPTG was added to the cell culture media.

Cells were harvested from the culture (400 ml) by centrifugation at 6,000 g for 20 min. Cell pellets were resuspended in 20 mM Tris-Cl, pH 8.0 (0.5 mM PMSF added 0.3 ml of 50 mM PMSF, 60 μl of 1 M 6-aminocaproic acid and 60 μl of 0.5 M benzamidine HCl in a final volume adjusted to 30 ml by adding the Tris buffer.

Cells were broken by passage through a French Press at 20,000 psi 3 times. The extracts were centrifuged at 10,000 rpm for 20 min. Supernatant was discarded and pellets (inclusion bodies) were resuspended in 20 ml of Tris buffer. Two ml of 20% Triton X-100 was added to each tube and sample volume was adjusted to 40 ml with Tris buffer. Tubes were incubated at room temperature for >1 hr while shaking and centrifuged at 10,000 rpm for 20 min. Supernatants were discarded and pellets were washed two times with Tris buffer by resuspending in 25 ml of Tris buffer and centrifugation and one time with 25 ml of pure water.

Solubilization of inclusion bodies was carried out as follows. Pellets were resuspended in 20 ml of water and 4 ml of 0.5 M CAPS buffer, pH 11, (50 mM final concentration), 40 μl of 1 M DTT (1 mM final conc.) and 0.4 ml of 30% sodium lauroyl Sarcosine (0.3% final conc.) were added. Sample volumes were adjusted to 40 ml with water. Samples were incubated at room temperature for 17 hr.

Refolding of the recombinant truncated arNOX was carried out as follows. After solubilization, the samples were centrifuged at 10,000 rpm for 20 min, and the supernatants were collected. The supernatants were filtered through a 0.45 μm nitrocellulose filter. The filtrates was poured into two dialysis bags (3500 MWCO, flat width 45 mm and diameter 29 mm, SpectraPor) and dialyzed against cold dialysis buffer 1 (25 mM Tris-HCl, pH 8.5, 1 mM cysteamine, 0.1 mM cyctamine, 1 mM 6-aminocaproic acid and 0.5 mM benzamidine HCl) with 3 changes, against cold dialysis buffer 2 (25 mM Tris-HCl, pH 8.0, 1 mM 6-aminocaproic acid and 0.5 mM benzamidine HCl) with one change and against dialysis buffer 3 (50 mM Tris-HCl, pH 8.0, 1 mM 6-aminocaproic acid and 0.5 mM benzamidine HCl) with one change. Dialysis was at least 17 hr following each change.

After dialysis, PMSF was added to a final concentration of 0.5 mM, and the sample was centrifuged at 10,000 rpm for 20 min. The supernatant was collected and concentrated to about 16 ml by using a Centriplus concentrator (Amicon, MWCO 10,000; 4700 rpm, 2800×g). Refolded arNOX was aliquoted to 0.5 ml into microcentrifuge tubes and stored at 80° C.

### Example 2

### Characterization of Recombinant arNOX

Reduction of ferric cytochrome c by superoxide was employed as a standard measure of superoxide formation (Mayo, L. A. and Curnutte, J., 1990, Meth. Enzymol. 186:567-575; Butler, J. et al., 1982, J. Biol. Chem. 257:10747-10750). This method, when coupled to superoxide dismutase inhibition, is generally accepted for the measurement of superoxide generation. The assay consists of 150 μl buffy coat material in PBSG buffer (8.06 g NaCl, 0.2 g KCl, 0.18 g Na₂HPO₄, 0.13 g CaCl₂, 0.1 g MgCl₂, 1.35 g glucose dissolved in 1000 ml deionized water, adjusted to pH 7.4, filtered and stored at 4° C.). Reduction of ferricytochrome c by superoxide was monitored as the increase in absorbance at 550 nm, with reference at 540 nm (Butler et al., 1982). As a further control for the specificity of the arNOX activity, 60 units of superoxide dismutase (SOD) were added near the end of the assay to ascertain that the rate returned to base line. Rates were determined using a SLM Aminco DW-2000 spectrophotometer in the dual wavelength mode of operation.

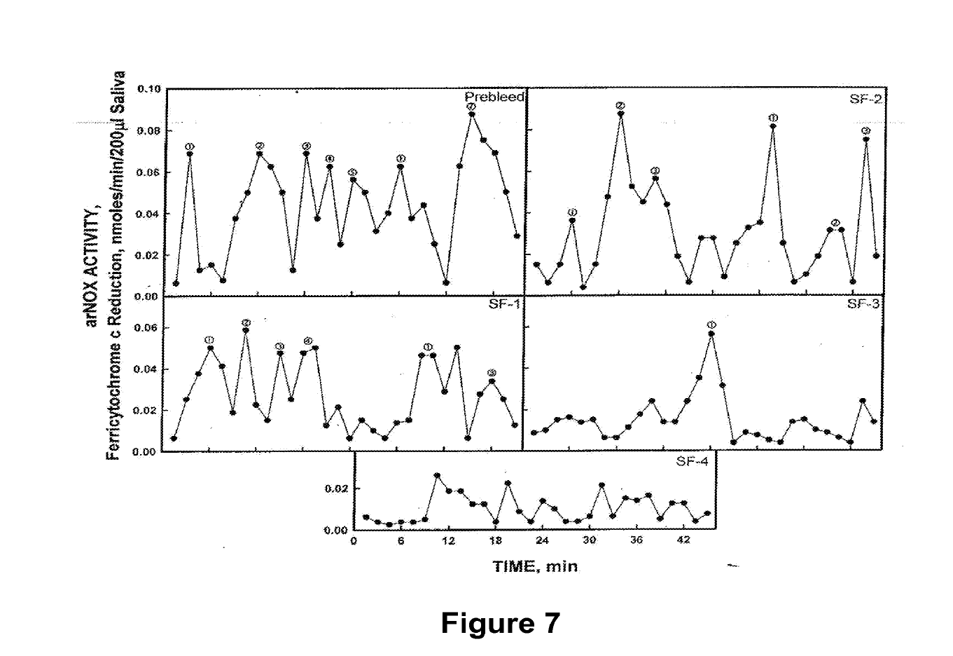

Rates were determined using an SLM Aminco DW-2000 spectrophotometer (Milton Roy, Rochester, N.Y.) in the dual wave length mode of operation with continuous measurements over 1 min every 1.5 min. After 45 min, test compounds were added and the reaction was continued for an additional 45 min. After 45 min, a millimolar extinction coefficient of 19.1 cm⁻¹ was used for reduced ferricytochrome c. The results of the test compounds are provided below (Table 4) for experiments carried out with TM9SF4, but from the results of FIG. 7, it is concluded that all the arNOX isoforms have similar responses to the various compounds given below. Extracts were made of the compounds in water unless otherwise indicated.

Table 4. Properties of Recombinant arNOX (TM9SF4)

26 min period resistant to similikalactone D
78% inhibited by superoxide dismutase
70% inhibited by arNOX inhibitor savory
80% inhibited by arNOX inhibitor gallic acid
70% inhibition by 3 way inhibitor (Dormin+Schizandra+Salicin)

All references cited herein are hereby incorporated by reference in their entireties to the extent they are not inconsistent with the present disclosure.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a Markush group or other grouping is used herein, all individual members of the group, and all combinations and subcombinations possible from the group, are intended to be individually included in the disclosure.

Every formulation or combination of components described or exemplified can be used to practice the invention, unless otherwise stated. Specific names of proteins or coding sequences or genes are intended to be exemplary, as it is known that one of ordinary skill in the art can name the same genes or proteins differently. When a compound is described herein such that a particular isoform of the protein is not specified, for example, that description is intended to include each isoform described individually or in any combination.

One of ordinary skill in the art will appreciate that vectors, promoters, coding methods, starting materials, synthetic methods, and the like other than those specifically exemplified can be employed in the practice of the invention without resort to undue experimentation. All art-known functional equivalents, of any such methods, vectors, promoters, coding sequences, synthetic methods, and the like are intended to be included in this description.

Whenever a range is given in the specification, for example, a temperature range, a time range, sequence relatedness range or a composition range, all intermediate ranges and subranges, as well as all individual values included in the ranges given are intended to be included herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of the appended claims.

As described herein, an aspect of the present disclosure concerns isolated nucleic acids and methods of use of isolated nucleic acids. In certain embodiments, the nucleic acid sequences disclosed herein and selected regions thereof have utility as hybridization probes or amplification primers. These nucleic acids may be used, for example, in diagnostic evaluation of tissue samples. In certain embodiments, these probes and primers consist of oligonucleotide fragments. Such fragments should be of sufficient length to provide specific hybridization to a RNA or DNA tissue sample. The sequences typically are 10-20 nucleotides, but may be longer. Longer sequences, e.g., 40, 50, 100, 500 and even up to full length, are preferred for certain embodiments.

Nucleic acid molecules having contiguous stretches of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75 80, 85, 90, 95, 100, 125, 150, 175, 200, 250, 300, 400, 500, 600, 750, 1000, 1500, 2000, 2500 or more nucleotides from a sequence selected from the disclosed nucleic acid sequences are contemplated. Molecules that are complementary to the above mentioned sequences and that bind to these sequences under high stringency conditions also are contemplated. These probes are useful in a variety of hybridization embodiments, such as Southern and Northern blotting.

The use of a hybridization probe of between 14 and 100 nucleotides in length allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained. One generally prefers to design nucleic acid molecules having stretches of 20 to 30 nucleotides, or even longer where desired. Such fragments may be readily prepared by, for example, directly synthesizing the fragment by chemical means or by introducing selected sequences into recombinant vectors for recombinant production.

Accordingly, the nucleotide sequences herein may be used for their ability to selectively form duplex molecules with complementary stretches of genes or RNAs or to provide primers for amplification of DNA or RNA from tissues. Depending on the application envisioned, one may desire to employ varying conditions of hybridization to achieve varying degrees of selectivity of probe towards target sequence.

For applications requiring high selectivity, one typically employs relatively stringent conditions to form the hybrids, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand, and would be particularly suitable for isolating specific genes or detecting specific mRNA transcripts. It is generally appreciated that conditions can be rendered more stringent by the addition of increasing amounts of formamide.

For certain applications, lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37 to about 55° C., while a low stringency condition could be provided by about 0.15 M to about 0.9 M salt, at temperatures ranging from about 20 to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. Other hybridization conditions utilized could include approximately 10 mM Tris-HCl (pH 8.3), 50 mM KCl, 1.5 μM MgCl₂, at temperatures ranging from approximately 40 to about 72° C.

In certain embodiments, it is advantageous to employ nucleic acid sequences as described herein in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known which can be employed to provide a detection means visible to the human eye or spectrophotometrically, to identify specific hybridization with complementary nucleic acid-containing samples.

In general, it is envisioned that the hybridization probes described herein are useful both as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The selected conditions depend on the particular circumstances based on the particular criteria required (depending, for example, on the G+C content, type of target nucleic acid, source of nucleic acid, size of hybridization probe, etc.). Following washing of the hybridized surface to remove non-specifically bound probe molecules, hybridization is detected, or quantified, by means of the label.

Methods disclosed herein are not limited to the particular probes disclosed and particularly are intended to encompass at least nucleic acid sequences that are hybridizable to the disclosed sequences or are functional sequence analogs of these sequences. For example, a partial sequence may be used to identify a structurally-related gene or the full length genomic or cDNA clone from which it is derived. Those of skill in the art are well aware of the methods for generating cDNA and genomic libraries which can be used as a target for the above-described probes (Sambrook et al., 1989).

For applications in which the nucleic acid segments of the present invention are incorporated into vectors, such as plasmids disclosed herein, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene after operably linking to the coding sequence of interest.

Where an expression product is to be generated, it is possible for the nucleic acid sequence to be varied while retaining the ability to encode the same product. Reference to a codon chart which provides synonymous coding sequences permits those of skill in the art to design any nucleic acid encoding for the polypeptide product of known amino acid sequence.

Plasmid preparations and replication means are well known in the art. See for example, U.S. Pat. Nos. 4,273,875 and 4,567,146.

Embodiments of the present invention include amplification of at least a portion of a target genetic material using conditions and reagents well known to the art.

Certain embodiments herein include any method for amplifying at least a portion of a microorganism's genetic material (such as Polymerase Chain Reaction (PCR), Real-time PCR (RT-PCR), NASBA (nucleic acid sequence based amplification)). In one embodiment, Real time PCR (RT-PCR) can be used for amplifying at least a portion of a subject's genetic material while simultaneously amplifying an internal control plasmid for verification of the outcome of the amplification of a subject's genetic material.

While the scope herein includes any method (for example, Polymerase Chain Reaction, i.e., PCR, and nucleic acid sequence based amplification, i.e., NASBA) for amplifying at least a portion of the microorganism's genetic material, for one example, the disclosure relates to embodiments in reference to a RT-PCR technique.

Typically, to verify the working conditions of PCR techniques, positive and negative external controls are performed in parallel reactions to the sample tubes to test the reaction conditions, for example using a control nucleic acid sequence for amplification. In some embodiments, an internal control can be used to determine if the conditions of the RT-PCR reaction is working in a specific tube for a specific target sample. Alternatively, in some embodiments, an internal control can be used to determine if the conditions of the RT-PCR reaction are working in a specific tube at a specific time for a specific target sample.

By knowing the nucleotide sequences of the genetic material in a subject mammal and in an internal control, specific primer sequences can be designed. In one embodiment of the present invention, at least one primer of a primer pair used to amplify a portion of genomic material of a target mammal is in common with one of the primers of a primer pair used to amplify a portion of genetic material of an internal control such as an internal control plasmid or other sequence of interest. In one embodiment, a primer is about, but not limited to 10 to 50 oligonucleotides long, or about 15 to 40 oligonucleotides long, or about 20 to 30 oligonucleotides long. Suitable primer sequences can be readily synthesized by one skilled in the art or are readily available from commercial providers such as BRL (New England Biolabs), etc. Other reagents, such as DNA polymerases and nucleotides, that are necessary for a nucleic acid sequence amplification such as PCR are also commercially available.

The presence or absence of PCR amplification product can be detected by any of the techniques known to one skilled in the art. In one particular embodiment, methods of the present invention include detecting the presence or absence of the PCR amplification product using a probe that hybridizes to a particular genetic material of the microorganism. By designing the PCR primer sequence and the probe nucleotide sequence to hybridize different portions of the microorganism's genetic material, one can increase the accuracy and/or sensitivity of the methods disclosed herein.

While there are a variety of labelled probes available, such as radioactive and fluorescent labelled probes, in one particular embodiment, methods use a fluorescence resonance energy transfer (FRET) labeled probe as internal hybridization probes. In a particular embodiment, an internal hybridization probe is included in the PCR reaction mixture so that product detection occurs as the PCR amplification product is formed, thereby reducing post-PCR processing time. Roche Lightcycler PCR instrument (U.S. Pat. No. 6,174,670) or other real-time PCR instruments can be used in this embodiment, e.g., see U.S. Pat. No. 6,814,934. In some instances, real-time PCR amplification and detection significantly reduce the total assay time. Accordingly, methods herein provide rapid and/or highly accurate results and these results are verified by an internal control.

In certain embodiments, DNA fragments can be introduced into the cells of interest by the use of a vector, which is a replicon in which another polynucleotide segment is attached, so as to bring the replication and/or expression to the attached segment. A vector can have one or more restriction endonuclease recognition sites at which the DNA sequences can be cut in a determinable fashion without loss of an essential biological function of the vector. Vectors can further provide primer sites (e.g. for PCR), transcriptional and/or translational initiation and/or regulation sites, recombinational signals, replicons, selectable markers, etc. Examples of vectors include plasmids, phages, cosmids, phagemid, yeast artificial chromosome (YAC), bacterial artificial chromosome (BAC), human artificial chromosome (HAG), virus, virus based vector, such as adenoviral vector, lentiviral vector, and other DNA sequences which are able to replicate or to be replicated in vitro or in a host cell, or to convey a desired DNA segment to a desired location within a host cell. The vector may be, for example, a phage, plasmid, viral, or retroviral vector. Retroviral vectors may be replication competent or replication defective. In the latter case, viral propagation generally will occur only in complementing host cells.

Polynucleotides may be joined to a vector containing a selectable marker for propagation in a host. If the vector is a virus, it may be packaged in vitro using an appropriate packaging cell line and then transduced into host cells.

Polynucleotide inserts may be operatively linked to an appropriate promoter, such as the phage lambda PL promoter, the E. coli lac, trp, phoA and tac promoters, the SV40 early and late promoters and promoters of retroviral LTRs, to name a few. Other suitable promoters are known to the skilled artisan. The expression constructs further contain sites for transcription initiation, termination, and, in the transcribed region, a ribosome binding site for translation. The coding portion of the transcripts expressed by the constructs preferably include a translation initiating codon at the beginning and a termination codon (UAA, UGA or UAG) appropriately positioned at the end of the polypeptide to be translated.

As indicated, the expression vectors can include at least one selectable marker. Exemplary markers can include, but are not limited to, dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera frugiperda Sf9 cells; animal cells such as CHO, COS, 293, and Bowes melanoma cells; and plant cells. Appropriate culture media, transformation techniques and conditions for cell growth and gene expression for the above-described host cells are known in the art.

In certain embodiments vectors of use for bacteria can include, but are not limited to, pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PA0815 (all available from Invitrogen, Carlbad, Calif.). Other suitable vectors are readily available to the art.

Recombinant DNA technologies used for the construction of the expression vector are those known and commonly used by persons skilled in the art. Standard techniques are used for cloning, isolation of DNA, amplification and purification; the enzymatic reactions involving DNA ligase, DNA polymerase, restriction endonucleases are carried out according to the manufacturer's recommendations. These techniques and others are generally carried out according to Sambrook et al. (1989).

In certain embodiments, an isolated host cell can contain a vector constructs described herein, and or an isolated host cell can contain nucleotide sequences herein that are operably linked to one or more heterologous control regions (e.g., promoter and/or enhancer) using techniques and sequences known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

It is contemplated herein that certain embodiments also encompasses primary, secondary, and immortalized host cells of vertebrate origin, particularly mammalian origin, that have been engineered to delete or replace endogenous genetic material (e.g., the coding sequence), and/or to include genetic material (e.g., heterologous polynucleotide sequences) that is operably associated with polynucleotides herein, and which activates, alters, and/or amplifies endogenous polynucleotides. For example, techniques known in the art may be used to operably associate heterologous control regions (e.g., promoter and/or enhancer) and endogenous polynucleotide sequences via homologous recombination (see, e.g., U.S. Pat. No. 5,641,670; WO 96/29411; WO 94/12650; Koller et al., Proc. Natl. Acad. Sci. USA 86:8932-8935 (1989); and Zijlstra et al., Nature 342:435-438 (1989).

Nucleic acids used as a template for amplification can be isolated from cells contained in the biological sample, according to standard methodologies. (Sambrook et al., 1989) The nucleic acid may be genomic DNA or fractionated or whole cell RNA. Where RNA is used, it may be desired to convert the RNA to a complementary cDNA. In one embodiment, the RNA is whole cell RNA and is used directly as the template for amplification.

Pairs of primers that selectively hybridize to nucleic acids corresponding to specific markers are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintilography of incorporated radiolabel or fluorescent label or even via a system using electrical or thermal impulse signals (Affymax, among others).

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641. Polymerase chain reaction methodologies are well known in the art. Other amplification methods are known in the art besides PCR such as LCR (ligase chain reaction), disclosed in European Publication No. 320 308).

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids herein. Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences may also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated. Still other amplification methods known in the art may be used with the methods described herein.

Following amplification, it may be desirable to separate the amplification product from the template and the excess primer for the purpose of determining whether specific amplification has occurred. Amplification products can be separated by agarose, agarose-acrylamide or polyacrylamide gel electrophoresis using standard methods. See Sambrook et al., 1989.

Alternatively, chromatographic techniques may be employed to effect separation of amplified product or other molecules. There are many kinds of chromatography which may be used: adsorption, partition, ion-exchange and molecular sieve, and many specialized techniques for using them including column, paper, thin-layer and gas chromatography, as known in the art.

Amplification products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products may then be exposed to x-ray film or visualized under the appropriate stimulating spectra, following separation.

Visualization can be achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, where the other member of the binding pair carries a detectable moiety.

In general, prokaryotes used for cloning DNA sequences in constructing the vectors useful herein can include but are not limited to, any gram negative bacteria such as E. coli strain K12 or strain W3110. Other microbial strains which may be used include P. aeruginosa strain PAO1, and E. coli B strain. These examples are illustrative rather than limiting. Other example bacterial hosts for constructing a library include but are not limited to, Escherichia, Pseudomonus, Salmonella, Serratia marcescens and Bacillus.

In general, plasmid vectors containing promoters and control sequences which are derived from species compatible with the host cell are used with these hosts. The vector ordinarily carries a replication site as well as one or more marker sequences which are capable of providing phenotypic selection in transformed cells. For example, a PBBR1 replicon region which is useful in many Gram negative bacterial strains or any other replicon region that is of use in a broad range of Gram negative host bacteria can be used in the present invention.

Promoters suitable for use with prokaryotic hosts illustratively include the β-lactamase and lactose promoter systems. In other embodiments, expression vectors used in prokaryotic host cells may also contain sequences necessary for efficient translation of specific genes encoding specific mRNA sequences that can be expressed from any suitable promoter. This would necessitate incorporation of a promoter followed by ribosomal binding sites or a Shine-Dalgarno (S.D.) sequence operably linked to the DNA encoding the mRNA.

Construction of suitable vectors containing the desired coding and control sequences employ standard ligation techniques. Isolated plasmids or DNA fragments are cleaved, tailored, and religated in the form desired to form the plasmids required.

For analysis to confirm correct sequences in plasmids constructed, the ligation mixtures are used to transform a bacteria strain such as E. coli K12 and successful transformants selected by antibiotic resistance such as tetracycline where appropriate. Plasmids from the transformants are prepared, analyzed by restriction and/or sequenced.

Isolated host cells can be transformed with expression vectors and cultured in conventional nutrient media modified as is appropriate for inducing promoters, selecting transformants or amplifying genes. The culture conditions, such as temperature, pH and the like, are those previously used with the host cell selected for expression, and will be apparent to the ordinarily skilled artisan.

Transformation refers to the taking up of an expression vector by a host cell whether or not any coding sequences are in fact expressed. Numerous methods for introducing a DNA molecule of interest into an isolated host cell are known to the art, for example, Ca salts and electroporation. Successful transformation is generally recognized when any indication of the operation of the vector occurs within the host cell.

Digestion of DNA refers to catalytic cleavage of the DNA with a restriction enzyme that acts only at certain sequences in the DNA. The various restriction enzymes used herein are commercially available and their reaction conditions, cofactors and other requirements were used as known to the art.

Recovery or isolation of a given fragment of DNA from a restriction digest means separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. This procedure is known generally (Lawn, R. et al., Nucleic Acids Res. 9: 6103 6114 [1981], and Goeddel, D. et al., Nucleic Acids Res. 8: 4057 [1980]).

Dephosphorylation refers to the removal of the terminal 5′ phosphates by treatment with bacterial alkaline phosphatase (BAP). This procedure prevents the two restriction cleaved ends of a DNA fragment from “circularizing” or forming a closed loop that would impede insertion of another DNA fragment at the restriction site. Procedures and reagents for dephosphorylation are conventional (Maniatis, T. et al., Molecular Cloning, 133-134, Cold Spring Harbor, [1982]). Reactions using BAP are carried out in 50 mM Tris at 68° C. to suppress the activity of any exonucleases which may be present in the enzyme preparations. Reactions are run for 1 hour. Following the reaction the DNA fragment is gel purified.

Ligation refers to the process of forming phosphodiester bonds between two double stranded nucleic acid fragments (Maniatis, T. et al., 1982, at 146). Unless otherwise provided, ligation may be accomplished using known buffers and conditions with 10 units of T4 DNA ligase (“ligase”) per 0.5 .mu.g of approximately equimolar amounts of the DNA fragments to be ligated.

Filling or blunting refers to the procedures by which the single stranded end in the cohesive terminus of a restriction enzyme-cleaved nucleic acid is converted to a double strand. This eliminates the cohesive terminus and forms a blunt end. This process is a versatile tool for converting a restriction cut end that may be cohesive with the ends created by only one or a few other restriction enzymes into a terminus compatible with any blunt-cutting restriction endonuclease or other filled cohesive terminus. In one embodiment, blunting is accomplished by incubating around 2 to 20 μg of the target DNA in 10 mM MgCl₂, 1 mM dithiothreitol, 50 mM NaCl, 10 mM Tris (pH 7.5) buffer at about 37° C. in the presence of 8 units of the Klenow fragment of DNA polymerase 1 and 250 μM of each of the four deoxynucleotide triphosphates. The incubation generally is terminated after 30 min. with phenol and chloroform extraction and ethanol precipitation

As used interchangeably herein, the terms “nucleic acid molecule(s)”, “oligonucleotide(s)”, and “polynucleotide(s)” include RNA or DNA (either single or double stranded, coding, complementary or antisense), or RNA/DNA hybrid sequences of more than one nucleotide in either single chain or duplex form (although each of the above species may be particularly specified). The term “nucleotide” is used herein as an adjective to describe molecules comprising RNA, DNA, or RNA/DNA hybrid sequences of any length in single-stranded or duplex form. More precisely, the expression “nucleotide sequence” encompasses the nucleic material itself and is thus not restricted to the sequence information (e.g. the succession of letters chosen among the four base letters) that biochemically characterizes a specific DNA or RNA molecule. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modifications such as (a) an alternative linking group, (b) an analogous form of purine, (c) an analogous form of pyrimidine, or (d) an analogous sugar. For examples of analogous linking groups, purine, pyrimidines, and sugars see for example, WO 95/04064, which disclosure is hereby incorporated by reference in its entirety. Preferred modifications of the present invention include, but are not limited to, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylguanosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylguanosine, 5′-methoxycarboxymethyluracil, 5-methoxyuracil, 2-methylthio-N6-isopentenyladenine, uracil-5-oxyacetic acid (v) ybutoxosine, pseudouracil, guanosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil, and 2,6-diaminopurine. The polynucleotide sequences herein may be prepared by any known method, including synthetic, recombinant, ex vivo generation, or a combination thereof, as well as utilizing any purification methods known in the art. Methylenemethylimino linked oligonucleotides as well as mixed backbone compounds, may be prepared as described in U.S. Pat. Nos. 5,378,825; 5,386,023; 5,489,677; 5,602,240; and 5,610,289. Formacetal and thioformacetal linked oligonucleotides may be prepared as described in U.S. Pat. Nos. 5,264,562 and 5,264,564. Ethylene oxide linked oligonucleotides may be prepared as described in U.S. Pat. No. 5,223,618. Phosphinate oligonucleotides may be prepared as described in U.S. Pat. No. 5,508,270. Alkyl phosphonate oligonucleotides may be prepared as described in U.S. Pat. No. 4,469,863. 3′-Deoxy-3′-methylene phosphonate oligonucleotides may be prepared as described in U.S. Pat. No. 5,610,289 or 5,625,050. Phosphoramidite oligonucleotides may be prepared as described in U.S. Pat. No. 5,256,775 or 5,366,878. Alkylphosphonothioate oligonucleotides may be prepared as described in WO 94/17093 and WO 94/02499. 3′-Deoxy-3′-amino phosphoramidate oligonucleotides may be prepared as described in U.S. Pat. No. 5,476,925. Phosphotriester oligonucleotides may be prepared as described in U.S. Pat. No. 5,023,243. Borano phosphate oligonucleotides may be prepared as described in U.S. Pat. Nos. 5,130,302 and 5,177,198.

The term “upstream” is used herein to refer to a location which is toward the 5′ end of the polynucleotide from a specific reference point.

The terms “base paired” and “Watson & Crick base paired” are used interchangeably herein to refer to nucleotides which can be hydrogen bonded to one another by virtue of their sequence identities in a manner like that found in double-helical DNA with thymine or uracil residues linked to adenine residues by two hydrogen bonds and cytosine and guanine residues linked by three hydrogen bonds.

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind. Unless otherwise stated, all complementary polynucleotides are fully complementary on the whole length of the considered polynucleotide.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides herein, although chemical or post-expression modifications of these polypeptides may be included excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination, as known to the art. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

As used herein, the terms “recombinant polynucleotide” and “polynucleotide construct” are used interchangeably to refer to linear or circular, purified or isolated polynucleotides that have been artificially designed and which comprise at least two nucleotide sequences that are not found as contiguous nucleotide sequences in their initial natural environment. In particular, these terms mean that the polynucleotide or cDNA is adjacent to “backbone” nucleic acid to which it is not adjacent in its natural environment. Backbone molecules according to the present invention include nucleic acids such as expression vectors, self-replicating nucleic acids, viruses, integrating nucleic acids, and other vectors or nucleic acids used to maintain or manipulate a nucleic acid insert of interest.

As used herein, the term “operably linked” refers to a linkage of polynucleotide elements in a functional relationship. A sequence which is “operably linked” to a regulatory sequence such as a promoter means that said regulatory element is in the correct location and orientation in relation to the nucleic acid to control RNA polymerase initiation and expression of the nucleic acid of interest. For instance, a promoter or enhancer is operably linked to a coding sequence if it affects the transcription of the coding sequence.

In one embodiment, the polynucleotides are at least 15, 30, 50, 100, 125, 500, or 1000 continuous nucleotides. In another embodiment, the polynucleotides are less than or equal to 300 kb, 200 kb, 100 kb, 50 kb, 10 kb, 7.5 kb, 5 kb, 2.5 kb, 2 kb, 1.5 kb, or 1 kb in length. In a further embodiment, polynucleotides herein comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides do not contain the coding sequence of more than 1000, 500, 250, 100, 75, 50, 25, 20, 15, 10, 5, 4, 3, 2, or 1 naturally occurring genomic flanking gene(s).

Procedures used to detect the presence of nucleic acids capable of hybridizing to the detectable probe include well known techniques such as Southern blotting, Northern blotting, dot blotting, colony hybridization, and plaque hybridization. In some applications, the nucleic acid capable of hybridizing to the labeled probe may be cloned into vectors such as expression vectors, sequencing vectors, or in vitro transcription vectors to facilitate the characterization and expression of the hybridizing nucleic acids in the sample. For example, such techniques may be used to isolate and clone sequences in a genomic library or cDNA library which are capable of hybridizing to the detectable probe as described herein.

Certain embodiments may involve incorporating a label into a probe, primer and/or target nucleic acid to facilitate its detection by a detection unit. A number of different labels may be used, such as Raman tags, fluorophores, chromophores, radioisotopes, enzymatic tags, antibodies, chemiluminescent, electroluminescent, affinity labels, etc. One of skill in the art recognizes that these and other label moieties not mentioned herein can be used in the disclosed methods.

Fluorescent labels of use may include, but are not limited to, Alexa 350, Alexa 430, AMCA (7-amino-4-methylcoumarin-3-acetic acid), BODIPY (5,7-dimethyl-4-bora-3a,4a-diaza-s-indacene-3-propionic acid) 630/650, BODIPY 650/665, BODIPY-FL (fluorescein), BODIPY-R6G (6-carboxyrhodamine), BODIPY-TMR (tetramethylrhodamine), BODIPY-TRX (Texas Red-X), Cascade Blue, Cy2 (cyanine), Cy3, Cy5,6-FAM (5-carboxyfluorescein), Fluorescein, 6-JOE (2′7′-di methoxy-4′5′-dichloro-6-carboxyfluorescein), Oregon Green 488, Oregon Green 500, Oregon Green 514, Pacific Blue, Rhodamine Green, Rhodamine Red, ROX (6-carboxy-X-rhodamine), TAMRA (N,N,N′,N′-tetramethyl-6-carboxyrhodamine), Tetramethylrhodamine, and Texas Red. Fluorescent or luminescent labels can be obtained from standard commercial sources, such as Molecular Probes (Eugene, Oreg.).

Examples of enzymatic labels include urease, alkaline phosphatase or peroxidase. Colorimetric indicator substrates can be employed with such enzymes to provide a detection means visible to the human eye or spectrophotometrically. Radioisotopes of potential use include ¹⁴C, ³H, ¹²⁵I, ³²P and ³⁵S.

In certain embodiments, expression vectors are employed to prepare materials for screening for inhibitors of one or more of the TM9SF arNOX isoforms. Expression can require appropriate signals be provided in the vectors, and which include various regulatory elements, such as enhancers/promoters from viral or mammalian sources that drive expression of the genes of interest in host cells. Bi-directional, host-factor independent transcriptional terminators elements may be incorporated into the expression vector and levels of transcription, translation, RNA stability or protein stability may be determined using standard techniques known in the art. The effect of the bi-directional, host-factor independent transcriptional terminators sequence may be determined by comparison to a control expression vector lacking the bidirectional, host-factor independent transcriptional terminators sequence, or to an expression vector containing a bidirectional, host-factor independent transcriptional terminators sequence of known effect.

In certain embodiments, an expression construct or expression vector, any type of genetic construct containing a nucleic acid coding for a gene product in which part or all of the nucleic acid coding sequence is capable of being transcribed, is constructed so that the coding sequence of interest is operably linked to and is expressed under transcriptional control of a promoter. A “promoter” refers to a DNA sequence recognized by the synthetic machinery of the cell, or introduced synthetic machinery, required to initiate the specific transcription of a gene. The phrase “under transcriptional control” can mean that the promoter is in the correct location and orientation in relation to the nucleic acid to control RNA polymerase initiation and expression of the gene in the isolated host cell of interest.

Where a cDNA insert is employed, typically one can include a polyadenylation signal to effect proper polyadenylation of the gene transcript. A terminator is also contemplated as an element of the expression construct. These elements can serve to enhance message levels and to minimize read through from the construct into other sequences.

In certain embodiments, the expression construct or vector contains a reporter gene whose activity may be detected or measured to determine the effect of a bi-directional, host-factor independent transcriptional terminators element or other element. Conveniently, the reporter gene produces a product that is easily assayed, such as a colored product, a fluorescent product or a luminescent product. Many examples of reporter genes are available, such as the genes encoding GFP (green fluorescent protein), CAT (chloramphenicol acetyltransferase), luciferase, GAL (β-galactosidase), GUS (β-glucuronidase), etc. The particular reporter gene employed is not important, provided it is capable of being expressed and expression can be detected. Further examples of reporter genes are well known to the art, and any of those known may be used in the practice of the claimed methods.

General references for cloning include Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989); Ausubel 1993, Current Protocols in Molecular Biology, Wiley, NY, among others readily available to the art.

Monoclonal or polyclonal antibodies specifically reacting with an arNOX protein of interest can be made by methods well known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) Current Protocols in Molecular Biology, Wiley Interscience/Greene Publishing, New York, N.Y., among others readily accessible to the art.

In general the terms and phrases used herein have their art-recognized meaning, which can be found by reference to standard texts, journal references and contexts known to those skilled in the art, unless otherwise defined.

What is claimed is:

1. A non-naturally occurring recombinant DNA molecule comprising a portion encoding a soluble aging-related NADH oxidase (arNOX) polypeptide or enzymatically active fragment thereof, said portion comprising a nucleotide sequence encoding a protein comprising an amino acid sequence selected from the group consisting of SEQ ID NO:2, SEQ ID NO:4, SEQ ID NO:6, SEQ ID NO:8, SEQ ID NO:10, SEQ ID NO:13, SEQ ID NO:14, SEQ ID NO:15, SEQ ID NO:16, and SEQ ID NO:17, or a nucleotide sequence which hybridizes under stringent conditions to one of the foregoing sequences and wherein said hybridizing sequence encodes an aging-related marker protein of the arNOX family of isoforms.

2. An isolated host cell transformed or transfected to contain the recombinant DNA molecules of claim 1.

3. The isolated host cell of claim 2 which is a bacterial cell.

4. The isolated host cell of claim 3 wherein said bacterial cell is an Escherichia coli cell.

5. The isolated host cell of claim 2 wherein said cell is a eukaryotic cell.

6. The isolated host cell of claim 2 wherein said cell is a mammalian cell.

7. The isolated host cell of claim 6 wherein said cell is a COS cell.

8. The isolated host cell of claim 5 wherein said cell is a yeast cell.

9. A method for recombinantly producing an arNOX active protein or polypeptide in a host cell, said method comprising the steps of:
a. infecting or transforming an isolated host cell with a vector comprising a promoter active in said host cell and a coding region for said arNOX polypeptide, wherein said arNOX protein or polypeptide comprises an amino acid sequence selected from the group consisting of the Transmembrane 9 superfamily members 1a, 1b, 2, 3 and 4 identified by amino acid sequences SEQ ID NO:2, SEQ ID NO:4, SEQ ID NO:6, SEQ ID NO:8, SEQ ID NO:10, SEQ ID NO:13, SEQ ID NO:14, SEQ ID NO:15, SEQ ID NO:16 or SEQ ID NO:17, said promoter being operably linked to said coding region, to produce a recombinant host cell; and.
b. culturing the recombinant host cell under conditions wherein said arNOX protein or polypeptide is expressed.

10. A method for determining aging status and arNOX isoform composition in a mammal, said method comprising the steps of:
a. providing a biological sample; and
b. detecting the presence in the biological sample, of a ribonucleic acid molecule encoding one or more arNOX proteins associated with aging, wherein the step of detecting is carried out using hybridization under stringent conditions or using a polymerase chain reaction in which a perfect match of primer to template is required, where a hybridization probe or primer consists essentially of at least 15 consecutive nucleotides of a nucleotide sequence as given in SEQ ID NO:1, SEQ ID NO:3, SEQ ID NO:5, SEQ ID NO:7 and SEQ ID NO:9;
wherein the presence of the ribonucleic acid molecule in the biological sample is indicative of arNOX expression.

11. An antibody preparation which specifically binds to an antigen selected from the group consisting of a protein characterized by an amino acid sequence as given in SEQ ID NO:2, SEQ ID NO:4, SEQ ID NO:6, SEQ ID NO:8, SEQ ID NO:10, SEQ ID NO:13, SEQ ID NO:14, SEQ ID NO:15, SEQ ID NO:16 or SEQ ID NO:17 or amino acids 56-87 of SEQ ID NO:2, amino acids 548-568 of SEQ ID NO:2, amino acids 73-104 of SEQ ID NO:6, amino acids 55-88 of SEQ ID NO:8 or amino acids 53-84 of SEQ ID NO:10.

12. A method for determining arNOX isoform compositions in a mammal, said method comprising the steps of:
a. providing a biological sample from a mammal;
b. contacting the biological sample of step a) with a detectable antibody specific for at least one arNOX protein under conditions which allow binding of the antibody to an arNOX protein; and
c. detecting the presence in a biological sample of at least one arNOX isoform the associated with aging-related disorders, when the detectable antibody specific the arNOX protein is bound.

13. An immunogenic composition effective in the amelioration of aging related disorders in a mammal, said composition comprising at least one arNOX protein or polypeptide set forth in SEQ ID NOs:2, 4, 6, 8, 10, 13, 14, 15, 16, or 17;
or a peptide having an amino acid sequence as given in amino acids 548-568 of SQ ID NO:2, amino acids 56-87 of SEQ ID NO:2, amino acids 73-104 of SEQ ID NO:6, amino acids 55-88 of SEQ ID NO:8 or amino acids 53-84 of SEQ ID NO:10.


SEQUENCE LISTING (17 sequences)

SEQ ID NO: 1 - DNA, 2449 bp, Homo sapiens
  CDS (349)..(2169)
ggccgcgctgccgatcgccgggaggacccccgcctcgccgaagacgggcggggcaagccgagcctcacggggtccccggagctgggccgggcctccagatggagaaggcgcaacggggagttcttgagtaagccagagcggtgtccagcgcggtgtagccgcagccgccgctgtcaggcgcagcaacgggcaaccccgtagaagtcggtcggcaggtcctctccaacccgccgctaccgcgccgctgtgggagagaccccagcaggagcccaaaggcagctacgggggcgcgaaggccgctggcgccgcctcggccagcccttcccgcgcggttccactgccttaaggatgacagtcgtagggaaccctcgaagttggagctgccagtggttgccaatcctgatactgttgctgggcacaggccatgggccaggggtggaaggcgtgacacactacaaggccggcgaccctgttattctgtatgtcaacaaagtgggaccctaccataaccctcaggaaacttaccactactatcagcttccagtctgctgccctgagaagatacgtcacaaaagccttagcctgggtgaagtgctggatggggaccgaatggctgagtctttgtatgagatccgctttcgggaaaacgtggagaagagaattctgtgccacatgcagctcagttctgcacaggtggagcagctgcgccaggccattgaagaactgtactactttgaatttgtggtagatgacttgccaatccggggctttgtgggctacatggaggagagtggtttcctgccacacagccacaagataggactctggacccatttggacttccacctagaattccatggagaccgaattatatttgccaatgtttcagtgcgggacgtcaagccccacagcttggatgggttacgacctgacgagttcctaggccttacccacacttatagcgtgcgctggtctgagacttcagtggagcgtcggagtgacaggcgccgtggtgacgatggtggtttctttcctcgaacactggaaatccattggttgtccatcatcaactccatggtgcttgtgtttttactggtgggttttgtggctgtcattctaatgcgtgtgcttcggaatgacctggctcggtacaacttagatgaggagaccacctctgcaggttctggtgatgactttgaccagggtgacaatggctggaaaattatccatacagatgtcttccgcttccccccataccgtggtctgctctgtgctgtgcttggcgtgggtgcccagttcctggcccttggcactggcattattgtcatggcactgctgggcatgttcaatgtgcaccgtcatggggccattaactcagcagccatcttgttgtatgccctgacctgctgcatctctggctacgtgtccagccacttctaccggcagattggaggcgagcgttgggtgtggaacatcattctcaccaccagtctcttctctgtgcctttcttcctgacgtggagtgtggtgaactcagtgcattgggccaatggttcgacacaggctctgccagccacaaccatcctgctgcttctgacggtttggctgctggtgggctttcccctcactgtcattggaggcatctttgggaagaacaacgccagcccctttgatgcaccctgtcgcaccaagaacatcgcccgggagattccaccccagccctggtacaagtctactgtcatccacatgactgttggaggcttcctgcctttcagtgccatctctgtggagctgtactacatctttgccacagtatggggtcgggagcagtacactttgtacggcatcctcttctttgtcttcgccatcctgctgagtgtgggggcttgcatctccattgcactcacctacttccagttgtctggggaggattaccgctggtggtggcgatctgtgctgagtgttggctccaccggcctcttcatcttcctctactcagttttctattatgcccggcgctccaacatgtctggggcagtacagacagtagagttcttcggctactccttactcactggttatgtcttcttcctcatgctgggcaccatctcctttttttcttccctaaagttcatccggtatatctatgttaacctcaagatggactgagttctgtatggcagaactattgctgttctctccctttcttcatgccctgttgaactctcctaccagcttctcttctgattgactgaattgtgtgatggcattgttgccttcccttttgccctttgggcattccttccccagagagggcctggaaattataaatctctatcacataaggattatatatttgaactttttaagttgcctttagttttggtcctgatttttctttttacaattaccaaaataaaatttattaagaaaaaggaaaaaaaaaaaa

SEQ ID NO: 2 - protein, 606 aa, Homo sapiens
MTVVGNPRSWSCQWLPILILLLGTGHGPGVEGVTHYKAGDPVILYVNKVGPYHNPQETYHYYQLPVCCPEKIRHKSLSLGEVLDGDRMAESLYEIRFRENVEKRILCHMQLSSAQVEQLRQAIEELYYFEFVVDDLPIRGFVGYMEESGFLPHSHKIGLWTHLDFHLEFHGDRIIFANVSVRDVKPHSLDGLRPDEFLGLTHTYSVRWSETSVERRSDRRRGDDGGFFPRTLEIHWLSIINSMVLVFLLVGFVAVILMRVLRNDLARYNLDEETTSAGSGDDFDQGDNGWKIIHTDVFRFPPYRGLLCAVLGVGAQFLALGTGIIVMALLGMFNVHRHGAINSAAILLYALTCCISGYVSSHFYRQIGGERWVWNIILTTSLFSVPFFLTWSVVNSVHWANGSTQALPATTILLLLTVWLLVGFPLTVIGGIFGKNNASPFDAPCRTKNIAREIPPQPWYKSTVIHMTVGGFLPFSAISVELYYIFATVWGREQYTLYGILFFVFAILLSVGACISIALTYFQLSGEDYRWWWRSVLSVGSTGLFIFLYSVFYYARRSNMSGAVQTVEFFGYSLLTGYVFFLMLGTISFFSSLKFIRYIYVNLKMD

SEQ ID NO: 3 - DNA, 2124 bp, Homo sapiens
  CDS (349)..(1818)
ggccgcgctgccgatcgccgggaggacccccgcctcgccgaagacgggcggggcaagccgagcctcacggggtccccggagctgggccgggcctccagatggagaaggcgcaacggggagttcttgagtaagccagagcggtgtccagcgcggtgtagccgcagccgccgctgtcaggcgcagcaacgggcaaccccgtagaagtcggtcggcaggtcctctccaacccgccgctaccgcgccgctgtgggagagaccccagcaggagcccaaaggcagctacgggggcgcgaaggccgctggcgccgcctcggccagcccttcccgcgcggttccactgccttaaggatgacagtcgtagggaaccctcgaagttggagctgccagtggttgccaatcctgatactgttgctgggcacaggccatgggccaggggtggaaggcgtgacacactacaaggccggcgaccctgttattctgtatgtcaacaaagtgggaccctaccataaccctcaggaaacttaccactactatcagcttccagtctgctgccctgagaagatacgtcacaaaagccttagcctgggtgaagtgctggatggggaccgaatggctgagtctttgtatgagatccgctttcgggaaaacgtggagaagagaattctgtgccacatgcagctcagttctgcacaggtggagcagctgcgccaggccattgaagaactgtactactttgaatttgtggtagatgacttgccaatccggggctttgtgggctacatggaggagagtggtttcctgccacacagccacaagataggactctggacccatttggacttccacctagaattccatggagaccgaattatatttgccaatgtttcagtgcgggacgtcaagccccacagcttggatgggttacgacctgacgagttcctaggccttacccacacttatagcgtgcgctggtctgagacttcagtggagcgtcggagtgacaggcgccgtggtgacgatggtggtttctttcctcgaacactggaaatccattggttgtccatcatcaactccatggtgcttgtgtttttactggtgggttttgtggctgtcattctaatgcgtgtgcttcggaatgacctggctcggtacaacttagatgaggagaccacctctgcaggttctggtgatgactttgaccagggtgacaatggctggaaaattatccatacagatgtcttccgcttccccccataccgtggtctgctctgtgctgtgcttggcgtgggtgcccagttcctggcccttggcactggcattattgtcatggcactgctgggcatgttcaatgtgcaccgtcatggggccattaactcagcagccatcttgttgtatgccctgacctgctgcatctctggctacgtgtccagccacttctaccggcagattggaggcgagcgttgggtgtggaacatcattctcaccaccagtctcttctctgtgcctttcttcctgacgtggagtgtggtgaactcagtgcattgggccaatggttcgacacaggctctgccagccacaaccatcctgctgcttctgacggtttggctgctggtgggctttcccctcactgtcattggaggcatctttgggaagaacaacgccagcccctttgatgcaccctgtcgcaccaagaacatcgcccgggagattccaccccagccctggtacaagtctactgtcatccacatgactgttggaggcttcctgcctttcaggtatcctccctttattccatggctattactgtcaggttcctgacctcaatttttcctgtccctactcatccagtaccctaacccaacccgttgatccctggttcagtggtaccattcagagatcattaaatggttcctcctatccccaagcaggactgagcttgaatgatatgagagtgtctcacttataaagctctccggagacatttcccccttcaccttcctggtttctgactttaatgcctatggacatcatgtggggtttaaagcccatttgatgacccatttactttgttgaatacctctttgtgccaggcaaagaataaagtggaataaaatggaaaaaaaa

SEQ ID NO: 4 - protein, 489 aa, Homo sapiens
MTVVGNPRSWSCQWLPILILLLGTGHGPGVEGVTHYKAGDPVILYVNKVGPYHNPQETYHYYQLPVCCPEKIRHKSLSLGEVLDGDRMAESLYEIRFRENVEKRILCHMQLSSAQVEQLRQAIEELYYFEFVVDDLPIRGFVGYMEESGFLPHSHKIGLWTHLDFHLEFHGDRIIFANVSVRDVKPHSLDGLRPDEFLGLTHTYSVRWSETSVERRSDRRRGDDGGFFPRTLEIHWLSIINSMVLVFLLVGFVAVILMRVLRNDLARYNLDEETTSAGSGDDFDQGDNGWKIIHTDVFRFPPYRGLLCAVLGVGAQFLALGTGIIVMALLGMFNVHRHGAINSAAILLYALTCCISGYVSSHFYRQIGGERWVWNIILTTSLFSVPFFLTWSVVNSVHWANGSTQALPATTILLLLTVWLLVGFPLTVIGGIFGKNNASPFDAPCRTKNIAREIPPQPWYKSTVIHMTVGGFLPFRYPPFIPWLLLSGS

SEQ ID NO: 5 - DNA, 2391 bp, Homo sapiens
  CDS (134)..(2125)
cgcaaccggaactagccttctgggggccggcttggtttatctctggcggccttgtagtcgtctccgagactccccacccctccttccctcttgaccccctaggtttgattgccctttccccgaaacaactatcatgagcgcgaggctgccggtgttgtctccacctcggtggccgcggctgttgctgctgtcgctgctcctgctgggggcggttcctggcccgcgccggagcggcgctttctacctgcccggcctggcgcccgtcaacttctgcgacgaagaaaaaaagagcgacgagtgcaaggccgaaatagaactatttgtgaacagacttgattcagtggaatcagttcttccttatgaatacacagcgtttgatttttgccaagcatcagaaggaaagcgcccatctgaaaatcttggtcaggtactattcggggaaagaattgaaccttcaccatataagtttacgtttaataagaaggagacctgtaagcttgtttgtacaaaaacataccatacagagaaagctgaagacaaacaaaagttagaattcttgaaaaaaagcatgttattgaattatcaacatcactggattgtggataatatgcctgtaacgtggtgttacgatgttgaagatggtcagaggttctgtaatcctggatttcctattggctgttacattacagataaaggccatgcaaaagatgcctgtgttattagttcagatttccatgaaagagatacattttacatcttcaaccatgttgacatcaaaatatactatcatgttgttgaaactgggtccatgggagcaagattagtggctgctaaacttgaaccgaaaagcttcaaacatacccatatagataaaccagactgctcagggccccccatggacataagtaacaaggcttctggggagataaaaattgcctatacttactctgttagcttcgaggaagatgataagatcagatgggcgtctagatgggactatattctggagtctatgcctcatacccacattcagtggtttagcattatgaattccctggtcattgttctcttcttatctggaatggtagctatgattatgttacggacactgcacaaagatattgctagatataatcagatggactctacggaagatgcccaggaagaatttggctggaaacttgttcatggtgatatattccgtcctccaagaaaagggatgctgctatcagtctttctaggatccgggacacagattttaattatgacctttgtgactctatttttcgcttgcctgggatttttgtcacctgccaaccgaggagcgctgatgacgtgtgctgtggtcctgtgggtgctgctgggcacccctgcaggctatgttgctgccagattctataagtcctttggaggtgagaagtggaaaacaaatgttttattaacatcatttctttgtcctgggattgtatttgctgacttctttataatgaatctgatcctctggggagaaggatcttcagcagctattccttttgggacactggttgccatattggccctttggttctgcatatctgtgcctctgacgtttattggtgcatactttggttttaagaagaatgccattgaacacccagttcgaaccaatcagattccacgtcagattcctgaacagtcgttctacacgaagcccttgcctggtattatcatgggagggattttgccctttggctgcatctttatacaacttttcttcattctgaatagtatttggtcacaccagatgtattacatgtttggcttcctatttctggtgtttatcattttggttattacctgttctgaagcaactatacttctttgctatttccacctatgtgcagaggattatcattggcaatggcgttcattccttacgagtggctttactgcagtttatttcttaatctatgcagtacactacttcttttcaaaactgcagatcacgggaacagcaagcacaattctgtactttggttataccatgataatggttttgatcttctttctttttacaggaacaattggcttctttgcatgcttttggtttgttaccaaaatatacagtgtggtgaaggttgactgaagaagtccagtgtgtccagttaaaacagaaataaattaaactcttcatcaacaaagacctgtttttgtgactgccttgagttttatcagaattattggcctagtaatccttcagaaacaccgtaattctaaataaacctcttcccatacacctttcccccataagatctgtcttcaacactataaagcatttgtattgtgatttgattaagtatatatttggttgttctcaatgaagagcaaatttaaatattatgtgcatttgaa

SEQ ID NO: 6 - protein, 663 aa, Homo sapiens
MSARLPVLSPPRWPRLLLLSLLLLGAVPGPRRSGAFYLPGLAPVNFCDEEKKSDECKAEIELFVNRLDSVESVLPYEYTAFDFCQASEGKRPSENLGQVLFGERIEPSPYKFTFNKKETCKLVCTKTYHTEKAEDKQKLEFLKKSMLLNYQHHWIVDNMPVTWCYDVEDGQRFCNPGFPIGCYITDKGHAKDACVISSDFHERDTFYIFNHVDIKIYYHVVETGSMGARLVAAKLEPKSFKHTHIDKPDCSGPPMDISNKASGEIKIAYTYSVSFEEDDKIRWASRWDYILESMPHTHIQWFSIMNSLVIVLFLSGMVAMIMLRTLHKDIARYNQMDSTEDAQEEFGWKLVHGDIFRPPRKGMLLSVFLGSGTQILIMTFVTLFFACLGFLSPANRGALMTCAVVLWVLLGTPAGYVAARFYKSFGGEKWKTNVLLTSFLCPGIVFADFFIMNLILWGEGSSAAIPFGTLVAILALWFCISVPLTFIGAYFGFKKNAIEHPVRTNQIPRQIPEQSFYTKPLPGIIMGGILPFGCIFIQLFFILNSIWSHQMYYMFGFLFLVFIILVITCSEATILLCYFHLCAEDYHWQWRSFLTSGFTAVYFLIYAVHYFFSKLQITGTASTILYFGYTMIMVLIFFLFTGTIGFFACFWFVTKIYSVVKVD

SEQ ID NO: 7 - DNA, 6140 bp, Homo sapiens
  CDS (218)..(1987)
gaggaagaggctgaggaggcgcggggggcgggggaggctcaggagcgggcggtgacggcgacggcggcggcagaggaggcagcggctgggccgggccccgtgcgtctgtccgcgccccgtggatgcgaatcggccgcggcggaggcggcggcggcggaggaggcggcggcgggaggaggagtcggtgagccggctccgggccggaggggcgcggaggatgaggccgctgcctggcgctcttggcgtggcggcggccgccgcgctgtggctgctgctgctgctgctgccccggacccgggcggacgagcacgaacacacgtatcaagataaagaggaagttgtcttatggatgaatactgttgggccctaccataatcgtcaagaaacatataagtacttttcacttccattctgtgtggggtcaaaaaaaagtatcagtcattaccatgaaactctgggagaagcacttcaaggggttgaattggaatttagtggtctggatattaaatttaaagatgatgtgatgccagccacttactgtgaaattgatttagataaagaaaagagagatgcatttgtatatgccataaaaaatcattactggtaccagatgtacatagatgatttaccaatatggggtattgttggtgaggctgatgaaaatggagaagattactatctttggacctataaaaaacttgaaataggttttaatggaaatcgaattgttgatgttaatctaactagtgaaggaaaggtgaaactggttccaaatactaaaatccagatgtcatattcagtaaaatggaaaaagtcagatgtgaaatttgaagatcgatttgacaaatatcttgatccgtccttttttcaacatcggattcattggttttcaattttcaactccttcatgatggtgatcttcttggtgggcttagtttcaatgattttaatgagaacattaagaaaagattatgctcggtacagtaaagaggaagaaatggatgatatggatagagacctaggagatgaatatggatggaaacaggtgcatggagatgtatttagaccatcaagtcacccactgatattttcctctctgattggttctggatgtcagatatttgctgtgtctctcatcgttattattgttgcaatgatagaagatttatatactgagaggggatcaatgctcagtacagccatatttgtctatgctgctacgtctccagtgaatggttattttggaggaagtctgtatgctagacaaggaggaaggagatggataaagcagatgtttattggggcattccttatcccagctatggtgtgtggcactgccttcttcatcaatttcatagccatttattaccatgcttcaagagccattccttttggaacaatggtggccgtttgttgcatctgtttttttgttattcttcctctaaatcttgttggtacaatacttggccgaaatctgtcaggtcagcccaactttccttgtcgtgtcaatgctgtgcctcgtcctataccggagaaaaaatggttcatggagcctgcggttattgtttgcctgggtggaattttaccttttggttcaatctttattgaaatgtatttcatcttcacgtctttctgggcatataagatctattatgtctatggcttcatgatgctggtgctggttatcctgtgcattgtgactgtctgtgtgactattgtgtgcacatattttctactaaatgcagaagattaccggtggcaatggacaagttttctctctgctgcatcaactgcaatctatgtttacatgtattccttttactactattttttcaaaacaaagatgtatggcttatttcaaacatcattttactttggatatatggcggtatttagcacagccttggggataatgtgtggagcgattggttacatgggaacaagtgcctttgtccgaaaaatctatactaatgtgaaaattgactagagacccaagaaaacctggaactttggatcaatttctttttcataggggtggaacttgcacagcaaaaacaaacaaacgcaagaagagatttgggctttaacacactgggtactttgtgggtctctctttcgtcggtggcttaaagtaacatctatttccattgatcctaggttcttcctgactgctttctccaactgttcacagcaaatgcttggattttatgcagtaggcattactacagtacatggctaatcttcccaaaaactagctcattaaagatgaaatagaccagctctcttcagtgaagaggacaaatagtttatttaaagcatttgttccaataaaataaatagagggaaacttggatgctaaaattacatgaataggaatcttcctggcacttagtgtttctatgttattgaaaaatgatgttccagaaagattacttttttcctcttatttttactgccattgtcgacctattgtgggacatttttatatattgaatctgggttcttttttgactttttttttttcccaatccaacagcatccttttttttaaaagagagaattagaaaatattaaatcctgcatgtaatatatctgctgtcatcttagttggaccaacttcccatttatttatcttaaaactatacagttacatcttaattccatccaaagaagatacagtttgaagacagaagtgtactctctacaatgcaatttactgtacagttagaaagcaaagtgttaaatggagaagatacttgtttttattaaacattttgagatttagataaactacattttaactgaatgtctaaagtgattatcttttttccccccaagttagtcttaaatcttttgggtttgaatgaaggttttacataagaaattattaaaaacaaggggggtgggtaataaatgtatataacattaaataatgtaacgtaggtgtagattcccaaatgcatttggatgtacagatcgactacagagtacttttttcttatgatgattggtgtagaaatgtgtgatttgggtgggcttttacatcttgcctaccattgcatgaaacattggggtttcttcaaaatgtgtgtgtcatacttcttttgggaggggggttgttttcttctgtttattttctgagactcctacaggagccaaatttgtaatttagagacacttaattttgttaatcctgtctgggacacttaagtaacatctaaagcattattgctttagaatgttcaaataaaatttcctgaccaaattgttttgtggaaatagatgtgtttgcaatttgaagatatctttctgtccagaaggcaaaattaccgaatgccatttttaaaagtatgctataaactatgctactctcatacaggggacccgtattttaaaatctccagacttgcttacatctagattatccagcacaatcataaagtgaatgacaaaccctttgaatgaaattgtggcacaaaatctgttcaggttggtgtaccgtgtaaagtggggatggggtaaaagtggttaacgtactgttggatcaacaaataaaggttacagttttgtaagagaagtgatttgaatacatttttctggaactattcataatatgaagttttcctagaaccactgagtttctagtttaatagtttgctatgcaaatgaccacctaaaacaatactttatattgttatttttagaaagactcaaaacacctgtatttaaaccttaatatgaaaatcatgcaattaatagttacacaagatgttttcattacaaaatatgtacctatctattgatggactctacatcctatattgtgacatgtaagtcctttaaaaggtgaaaagtatgatttcttaccacttaagtatgattgatatgatccaacaaatttgatcagaagctgtaggtaaatcctcttctgaagccaaaatggtatattaaatataatttattggtacttccattttctcttccttcttacttgcctttaagatcttataaaaaagaaactaaaagttaatatttagttgcctatattatgtaaccttttaactatatataaagtacttttttggtttctttctcaccacttttattcaaaagtacttttaacataccaatacatagtctgtctgatgggagtataaattggacagtaaggttttgtcttaataaaatgaaatttgtttctcatgatatgaatcttgcaggtaagatgtagggtttattgaaaatgtgtgggttaaatgctttcaggtacaccaattctttctactaaattgagctctatttgaagttctttggaatctgtggtgaaaaataattttctgatttccaaatacattaagagcattaaatgaatattaatcacctttaaagtcttttagaaaaggacttgtattggtttttggctgcatagaggggttgaataagtgtatgtatgtgtgtgcgtgtgtgtgtgtcttcttaaagaagatgtaattcacaaatagtttagctccctagcgctcagttgtagaatagaaaatagaacattattcaagttaattgaaaggtgaggtttttatacccccactaatgctgtgtatctgtctttcgtttgttaacattatttgcttaatttctttcaactcacactttggataatactatcaaaaactaaggctaaacattccttgtgtatctttaagcatgcttctcctgaaatttaactacattagtagttgacatttgtatacatatatcctaatacaagagtaggataaggtggaaatgtaatggcctgagggatggtgaagcattcttttagtatttttcatcatgttgggctcctagattgtactggggttgcccataaatcaaaccccatactcttagaattcattatattatggtgatatccgaacctagtgaatggtatgcttgggtgttttccattgagagtggatggacctctttataaagttggttgctgcaaaatccagttcttccaaaagccactttatttagggtttattcacaagtcatatccattttggtacagtgtttgtttcctaatatttattaaccaccttataccaaatgtcttgcaaagaaatgttattaaaaccttgaatttttacaaatgtaaaaaacaaaaagtgtattaatgtatttgttcaggaaaagctacataccgaagggcttttgtatatgaattctgtggtggggagacccatttgtaatctatatggcagttccatctgggttttaagtttagatttcaccgtgtcttagtgcttcattctattggtttattggaacatgtaataaataggagtagtgatgtattaaaacacaagtattcattaatgttttatatcttcactaaaattctatagttatgaaactatcaatcaaggtgttatatttcagtcagaagtgaaaatttatgaagagtatttggaagtgtgtacagaaataaactagacttacaggtaggctagatcagaacgttaacatatgaacctgcagaaatctggtaagacttaaattcagtgtgaggaataactctagttctctcctatgagcatttcctaaaagccatctgatttggcattcttactggagctgcagacagaaatctacaaagacaaaagtaaacaaaattaagttattattccactgttaggaatggaaataaacttgtgaagtctgtttattttgaagtattggtgaactaggcttgctaattgataactgcagcagtttgtgtttactccagttcatcagcttaggtcatttgaaagatataagagcttaaggcaagaaagaaataacatggaattctatttgaaggacaacagaacattcttggaaaagcagctccagttggtttttcaactgtcaaacttgaatgtgtaagtccccacagagcatggacagtcggtgcagagttccaaggaaacaattattgcctgatgaccacttccattttgtatacactctttggttcgtataggccatattccaactggctttttagtaatagaaatccagtatataatgtatcaaatacaattgaggttctaacctagtgtgttaatttatctgaatttggatttttaaaaagtaataaaaagttaaatgta

SEQ ID NO: 8 - protein, 589 aa, Homo sapiens
MRPLPGALGVAAAAALWLLLLLLPRTRADEHEHTYQDKEEVVLWMNTVGPYHNRQETYKYFSLPFCVGSKKSISHYHETLGEALQGVELEFSGLDIKFKDDVMPATYCEIDLDKEKRDAFVYAIKNHYWYQMYIDDLPIWGIVGEADENGEDYYLWTYKKLEIGFNGNRIVDVNLTSEGKVKLVPNTKIQMSYSVKWKKSDVKFEDRFDKYLDPSFFQHRIHWFSIFNSFMMVIFLVGLVSMILMRTLRKDYARYSKEEEMDDMDRDLGDEYGWKQVHGDVFRPSSHPLIFSSLIGSGCQIFAVSLIVIIVAMIEDLYTERGSMLSTAIFVYAATSPVNGYFGGSLYARQGGRRWIKQMFIGAFLIPAMVCGTAFFINFIAIYYHASRAIPFGTMVAVCCICFFVILPLNLVGTILGRNLSGQPNFPCRVNAVPRPIPEKKWFMEPAVIVCLGGILPFGSIFIEMYFIFTSFWAYKIYYVYGFMMLVLVILCIVTVCVTIVCTYFLLNAEDYRWQWTSFLSAASTAIYVYMYSFYYYFFKTKMYGLFQTSFYFGYMAVFSTALGIMCGAIGYMGTSAFVRKIYTNVKID

SEQ ID NO: 9 - DNA, 3996 bp, Homo sapiens
  CDS (236)..(2164)
agtttctgccaggagctaatatggcttccttagttacaccgttctctctcttcacctaatcagcgaccttactttcccagaccagactgtcgagcaggagctaagactccttttcccctctgctgaccgccactacaggagcggttgaagccagacgaccaccttgtggagttaaactccgtaaccagggagcaccacttccgctgacgtcattacggcgacacgtggatccaagatggcgacggcgatggattggttgccgtggtctttactgcttttctccctgatgtgtgaaacaagcgccttctatgtgcctggggtcgcgcctatcaacttccaccagaacgatcccgtagaaatcaaggctgtgaagctcaccagctctcgaacccagctaccttatgaatactattcactgcccttctgccagcccagcaagataacctacaaggcagagaatctgggagaggtgctgagaggggaccggattgtcaacacccctttccaggttctcatgaacagcgagaagaagtgtgaagttctgtgcagccagtccaacaagccagtgaccctgacagtggagcagagccgactcgtggccgagcggatcacagaagactactacgtccacctcattgctgacaacctgcctgtggccacccggctggagctctactccaaccgagacagcgatgacaagaagaaggaaaaagatgtgcagtttgaacacggctaccggctcggcttcacagatgtcaacaagatctacctgcacaaccacctctcattcatcctttactatcatcgggaggacatggaagaggaccaggagcacacgtaccgtgtcgtccgcttcgaggtgattccccagagcatcaggctggaggacctcaaagcagatgagaagagttcgtgcactctgcctgagggtaccaactcctcgccccaagaaattgaccccaccaaggagaatcagctgtacttcacctactctgtccactgggaggaaagtgatatcaaatgggcctctcgctgggacacttacctgaccatgagtgacgtccagatccactggttttctatcattaactccgttgttgtggtcttcttcctgtcaggtatcctgagcatgattatcattcggaccctccggaaggacattgccaactacaacaaggaggatgacattgaagacaccatggaggagtctgggtggaagttggtgcacggcgacgtcttcaggcccccccagtaccccatgatcctcagctccctgctgggctcaggcattcagctgttctgtatgatcctcatcgtcatctttgtagccatgcttgggatgctgtcgccctccagccggggagctctcatgaccacagcctgcttcctcttcatgttcatgggggtgtttggcggattttctgctggccgtctgtaccgcactttaaaaggccatcggtggaagaaaggagccttctgtacggcaactctgtaccctggtgtggtttttggcatctgcttcgtattgaattgcttcatttggggaaagcactcatcaggagcggtgccctttcccaccatggtggctctgctgtgcatgtggttcgggatctccctgcccctcgtctacttgggctactacttcggcttccgaaagcagccatatgacaaccctgtgcgcaccaaccagattccccggcagatccccgagcagcggtggtacatgaaccgatttgtgggcatcctcatggctgggatcttgcccttcggcgccatgttcatcgagctcttcttcatcttcagtgctatctgggagaatcagttctattacctctttggcttcctgttccttgttttcatcatcctggtggtatcctgttcacaaatcagcatcgtcatggtgtacttccagctgtgtgcagaggattaccgctggtggtggagaaatttcctagtctccgggggctctgcattctacgtcctggtttatgccatcttttatttcgttaacaagctggacatcgtggagttcatcccctctctcctctactttggctacacggccctcatggtcttgtccttctggctgctaacgggtaccatcggcttctatgcagcctacatgtttgttcgcaagatctatgctgctgtgaagatagactgattggagtggaccacggccaagcttgctccgtcctcggacaggaagccaccctgcgtgggggactgcaggcacgcaaaataaaataactcctgctcgtttggaatgtaactcctggcacagtgttcctggatcctggggctgcgtggggggcgggagggcctgtagataatcttgcgtttttcgtcatcttattccagttctgtgggggatgagtttttttgtgggttgctttttcttcagtgctaagaaagttccctccaacaggaactctctgacctgtttattcaggtgtatttctggtttggatttttttttccttctttgttttaacaaatggatccaggatggataaatccaccgagataagggttttggtcactgtctccacctcagttcctcagggctgttggccaccctatgactaactggaagaggacacgccagagcttcagtgaggtttccgagcctctccctgcccatcctcaccactgaggccacgacaaagcacagctccagctcggacagcaccctcagtgccagccagcctctgccagacctctctttccctcttctccccagcctcctccagggctgcccaaggcagggtttccagccaggcctcggggtcatcttttcaccaggagcaaacccaagtcttagttgctacaagaaaatcccctggaagtactgggggccaggttccccagacagcaggaattgcccctgttcagagcagccggagtttgctggaccacaaggaagaagagaagagacttgcagtgaactgtttttgtgccaagaaaccctggacctggggccaagtatttcccaagccaagcatccacttgtctgtgtctgggaagggatggccaaggccgctagggtccttacccctcaggatcactccccagccctttcctcaggaggtaccgctctccaaggtgtgctagcagtgggccctgcccaacttcaggcagaacagggaggcccagagattacagatcccctcctgtaagtggccaggcattctctccctgccctctctggcctctggggtcatactcacttctttagccagccccatcccctccaccccacacctgagttcttgcctcctccttttggggacacccaaaacactgcttgtgagaaggaagatggaaggtaagttctgtcgttctttccccaatccccaggaatggacaagaagccaacttagaaagaagggtctcacgtggctggcctggctcctccgtagacccctgttcttttcaacctctgcccacccgtgcatgtcatcacaaacatttgctcttaagttacaagagaccacatccacccagggattagggttcaagtagcagctgctaacccttgcaccagcccttgtgggactcccaacacaagacaaagctcaggatgctggtgatgctaggaagatgtccctcccctcactgccccacattctcccagtggctctaccagcctcacccatcaaaccagtgaatttctcaatcttgcctcacagtgactgcagcgccaagcggcatccaccaagcatcaagttggagaaaagggaacccaagcagtagagagcgatattggagtcttttgttcattcaaatcttggatttttttttttccctaagagattctctttttagggggaatgggaaacggacacctcataaagggttcaaagatcatcaatttttctgactttttaaatcattatcattattatttttaattaaaaaaatgcctgtatgcctttttttggtcggattgtaaataaatataccattgtcctactgaaaaaaaaaaaaaaaaaa

SEQ ID NO: 10 - protein, 642 aa, Homo sapiens
MATAMDWLPWSLLLFSLMCETSAFYVPGVAPINFHQNDPVEIKAVKLTSSRTQLPYEYYSLPFCQPSKITYKAENLGEVLRGDRIVNTPFQVLMNSEKKCEVLCSQSNKPVTLTVEQSRLVAERITEDYYVHLIADNLPVATRLELYSNRDSDDKKKEKDVQFEHGYRLGFTDVNKIYLHNHLSFILYYHREDMEEDQEHTYRVVRFEVIPQSIRLEDLKADEKSSCTLPEGTNSSPQEIDPTKENQLYFTYSVHWEESDIKWASRWDTYLTMSDVQIHWFSIINSVVVVFFLSGILSMIIIRTLRKDIANYNKEDDIEDTMEESGWKLVHGDVFRPPQYPMILSSLLGSGIQLFCMILIVIFVAMLGMLSPSSRGALMTTACFLFMFMGVFGGFSAGRLYRTLKGHRWKKGAFCTATLYPGVVFGICFVLNCFIWGKHSSGAVPFPTMVALLCMWFGISLPLVYLGYYFGFRKQPYDNPVRTNQIPRQIPEQRWYMNRFVGILMAGILPFGAMFIELFFIFSAIWENQFYYLFGFLFLVFIILVVSCSQISIVMVYFQLCAEDYRWWWRNFLVSGGSAFYVLVYAIFYFVNKLDIVEFIPSLLYFGYTALMVLSFWLLTGTIGFYAAYMFVRKIYAAVKID

SEQ ID NO: 11 - DNA, 36 bp, Artificial Sequence
  Synthetic construct  oligonucleotide useful as a primer.
gatatacatatggctagcatggcgacggcgatggat

SEQ ID NO: 12 - DNA, 36 bp, Artificial Sequence
  Synthetic construct  oligonucleotide useful as a primer.
ttgttagcagccggatcctcagtctatcttcacagc

SEQ ID NO: 13 - protein, 237 aa, Homo sapiens
MTVVGNPRSWSCQWLPILILLLGTGHGPGVEGVTHYKAGDPVILYVNKVGPYHNPQETYHYYQLPVCCPEKIRHKSLSLGEVLDGDRMAESLYEIRFRENVEKRILCHMQLSSAQVEQLRQAIEELYYFEFVVDDLPIRGFVGYMEESGFLPHSHKIGLWTHLDFHLEFHGDRIIFANVSVRDVKPHSLDGLRPDEFLGLTHTYSVRWSETSVERRSDRRRGDDGGFFPRTLEIHWL

SEQ ID NO: 14 - protein, 237 aa, Homo sapiens; identical to SEQ ID NO: 13

SEQ ID NO: 15 - protein, 250 aa, Homo sapiens
MSARLPVLSPPRWPRLLLLSLLLLGAVPGPRRSGAFYLPGLAPVNFCDEEKKSDECKAEIELFVNRLDSVESVLPYEYTAFDFCQASEGKRPSENLGQVLFGERIEPSPYKFTFNKKETCKLVCTKTYHTEKAEDKQKLEFLKKSMLLNYQHHWIVDNMPVTWCYDVEDGQRFCNPGFPIGCYITDKGHAKDACVISSDFHERDTFYIFNHVDIKIYYHVVETGSMGARLVAAKLEPKSFKHTHIDKPDC

SEQ ID NO: 16 - protein, 240 aa, Homo sapiens
MRPLPGALGVAAAAALWLLLLLLPRTRADEHEHTYQDKEEVVLWMNTVGPYHNRQETYKYFSLPFCVGSKKSISHYHETLGEALQGVELEFSGLDIKFKDDVMPATYCEIDLDKEKRDAFVYAIKNHYWYQMYIDDLPIWGIVGEADENGEDYYLWTYKKLEIGFNGNRIVDVNLTSEGKVKLVPNTKIQMSYSVKWKKSDVKFEDRFDKYLDPSFFQHRIHWFSIFNSFMMVIFLVGLV

SEQ ID NO: 17 - protein, 252 aa, Homo sapiens
MATAMDWLPWSLLLFSLMCETSAFYVPGVAPINFHQNDPVEIKAVKLTSSRTQLPYEYYSLPFCQPSKITYKAENLGEVLRGDRIVNTPFQVLMNSEKKCEVLCSQSNKPVTLTVEQSRLVAERITEDYYVHLIADNLPVATRLELYSNRDSDDKKKEKDVQFEHGYRLGFTDVNKIYLHNHLSFILYYHREDMEEDQEHTYRVVRFEVIPQSIRLEDLKADEKSSCTLPEGTNSSPQEIDPTKENQLYFTY